(19) United States
(12) Patent Application Publication
(10) Pub. No.: US 2015/0112411 A1
(43) Pub. Date: Apr. 23, 2015

(54) HIGH POWERED LIGHT EMITTING DIODE PHOTOBIOLOGY COMPOSITIONS, METHODS AND SYSTEMS

(71) Applicant: VARAYA PHOTOCEUTICALS, LLC, Irvine, CA (US)

(72) Inventors: FRANCES BECKMAN, IRVINE, CA (US); MYK LUM, IRVINE, CA (US)

(21) Appl. No.: 14/515,965

(22) Filed: Oct. 16, 2014

Related U.S. Application Data
(60) Provisional application No. 61/892,817, filed on Oct. 18, 2013.

(51) Int. Cl. A61N 5/06

(52) U.S. Cl. CPC A61N 5/06; A61N 2005/0651; A61N 2005/0663; A61N 2005/0659; A61N 2005/0626
USPC 607/90

(57) ABSTRACT

Devices with high-power light-emitting diodes (LEDs) for use in human and/or animal phototherapy applications are disclosed. The phototherapy device includes a number of select LEDs for emitting a desired range or ranges of wavelengths of high intensity light for use in treatment. Additionally, the phototherapy treatment includes one or more methods for providing a treatment appropriate to the condition desired to be treated. The phototherapy device provides a diversity of high power light settings, intensity levels, and selectable time intervals.

### CROSS-REFERENCE TO RELATED APPLICATIONS

This application relates to and claims the benefit of U.S. Provisional Application No. 61/892,817 filed Oct. 18, 2013 and entitled “HIGH POWERED LIGHT EMITTING DIODE PHOTOBIOLOGY COMPOSITIONS, METHODS and SYSTEMS” the disclosure of which is wholly incorporated by reference in its entirety herein.

### STATEMENT RE: FEDERALLY SPONSORED RESEARCH/DEVELOPMENT

Not Applicable

### BACKGROUND

1. Technical Field

The present disclosure generally relates to high-power light-emitting diodes (LEDs) for use in human and/or animal phototherapy applications, and more particularly, a phototherapy device including a number of select LEDs for emitting a desired range or ranges of wavelengths of high intensity light for use in treatment and having a diversity of high power light settings, intensity levels, and selectable time intervals. The disclosure also relates to phototherapy treatment appropriate for the condition to be treated.

2. Related Art

Phototherapy relates to the treatment of biological tissues using one or more ranges of light wavelengths including, for example, visible, ultraviolet, and/or infrared light. Compared with laser treatments, the intensity of the light used in phototherapy is much lower and does not require the levels of risk of laser emissions. Phototherapy consists of exposure to specific wavelengths of light using LEDs (i.e., as individual LEDs and/or arrays of LEDs) as light sources, with a prescribed intensity and amount of time to treat disease, provide symptomatic relief, and affect cosmetic enhancements to hair, skin and body. Phototherapy with LED devices studied in recent decades produce results that demonstrate photo-biomodulation. Phototherapy treatments using high-powered LED devices of the type set forth herein take advantage of the bio-stimulatory effects of the light energy produced. Light energy is composed of photons (i.e., discrete packets of electromagnetic energy). The energy dose of light varies with the number of photons and their wavelength or color. Photons delivered to living tissue may be scattered or absorbed. Scattered photons may be eventually absorbed by, or escape from, the subject tissue.

Photons that escape the subject tissue do so through the action of diffuse reflection. Absorbed photons may interact with one or more organic molecules and/or chromophores within the subject tissue. Interactions with the subject tissue produce photochemistry. Thus, appropriate controlled application of light is capable of producing beneficial results.

Use of phototherapy in clinical care and aesthetic applications is rapidly evolving and expanding.

More and more benefits are being revealed for applying selected wavelengths of light to various sections of tissue in order to stimulate cellular proficiency, and enhance the body's ability to heal and regenerate. Phototherapy finds beneficial applications in the treatment of acne, wrinkles, sun and age spots, rosacea, eczema, hair loss, and wound healing, symptomatic pain relief, and physical medicine rehabilitation. Beneficial ranges of light wavelength may overlap with each other in treating certain ailments, and serve to promote a variety of benefits to the hair, skin, and body. Light sources are often used in combination with varying degrees of stimulation to increase efficacy, but absorption has proven to be a key to cellular change. Phototherapy emits photons that are absorbed by photoreceptors in the skin and body. Photo-receptive cells can be stimulated at differing depths dependent upon wavelength and intensity. Hair and skin cells respond well to phototherapy involving low level light because the cells of these reside just beneath the skin surface, allowing use low levels of energy able to reach the receptor sites and induce desired photochemistry to achieve beneficial results.

A multitude of phototherapy devices is currently available for home or professional use to treat skin, body, and hair. However, existing devices suffer from a number of deficiencies. Professional units are often stationary, large, and cumbersome because of the number of LEDs necessary to achieve the desired light intensity. Consumer or personal devices are often underpowered and unable to provide an adequate number of LEDs in a handheld or other conveniently-sized unit. Existing handheld units are lacking in both the ability to deliver adequate light intensity and the selectability of an adequate range of wavelengths to achieve desired results. Moreover, existing phototherapy devices may not allow multiple wavelengths to be operated simultaneously, or have integrated optics.

U.S. Pat. No. 7,513,906 to Passy et al. discloses a phototherapy apparatus incorporating interconnected radiation sources for providing irradiation over time to aid in bone healing, growth, and regeneration. Like many similar devices, there are an excessive numbers of diodes, while limiting the convenience and versatility of the apparatus resulting from a limited range of light energy wavelengths.

U.S. Pat. No. 6,019,482 to Everett discloses a hand-held, self-contained irradiator powered by batteries. The irradiator provides an applicator having many diodes that emit electromagnetic radiation in the visible and/or infrared portions of the spectrum. By activating particular switches, different wavelengths can be emitted from the applicator end to treat particular body surface areas for the relief of pain or other problems. The Everett irradiator fails to deliver light energy levels adequate for the desired benefits, and in effort to generate adequate light, incorporates a large array of diodes that generates heat, and significantly reduces convenience of use and effectiveness.

U.S. Pat. No. 7,686,839 to Parker describes phototherapy treatment devices for applying close-proximity area lighting to a wound for providing light/heat energy to aid in healing, but does not provide the convenience and flexibility of use needed for a versatile and user-friendly device.

U.S. Pat. No. 7,198,634 to Harth et al. discloses the advantages of phototherapy for inducing the nitric oxide (NO) effect of dilating vascular walls, but does so within a limited infrared light source in combination with topical ingredients, thereby reducing the over-all effectiveness of such a procedure.

Existing LED phototherapy devices oftentimes utilize incorrect emission wavelengths. In addition, the LED power output power of existing devices is insufficient to sustain the beneficial effects of phototherapy, and therefore tend to be less effective, and even ineffective. Other conventional phototherapy devices may have sufficient LED power output, but are large and prohibitively expensive for self-use, thereby limiting their value in personal medical and aesthetic care. Rather, they require costly, time-consuming, and inconvenient trips to a medical office.

Accordingly, there is a need in the art for devices suitable for phototherapy of the skin and body to achieve improved cosmetic, medical, and psychological results. There is a need to incorporate a selected range and/or combination of light sources, wavelengths, frequencies (hertz), photon dosages and angles of incidence to achieve optimal photo-biological benefits, in a diversity of user-friendly configurations to allow for a range of professional and consumer applications.

### BRIEF SUMMARY

In accordance with one embodiment of the present disclosure, a portable high-powered light emitting diode photobiology device for treatment of biological tissues is contemplated. The device may include a plurality of light emitting diodes, including a first one having a first predetermined wavelength with a first emission axis, as well as a second one having a second predetermined wavelength with a second emission axis. Additionally, the device may have a plurality of optics including a first optic corresponding to the first one of the plurality of light emitting diodes that defines a first dispersion pattern of enhanced light intensity centered on the first emission axis. There may also be a second optic corresponding to the second one of the plurality of light emitting diodes that defines a second dispersion pattern of enhanced light intensity centered on the second emission axis. The device may further include an optical face defined by a flat planar surface. The first one of the plurality of light emitting diodes may be positioned in a first tilted angular relationship relative to the flat planar surface of the optical face. The second one of the plurality of light emitting diodes may be positioned in a second tilted angular relationship relative to the flat planar surface of the optical face. The first emission axis and the second emission axis may intersect at a predefined distance away from the optical face and define a substantially overlapping emission region of the first dispersion pattern and the second dispersion pattern.

Another embodiment of the present disclosure is directed to a portable, high-powered light emitting diode photobiology device for use in phototherapy applications and treatment of biological tissues. The device may include a plurality of light emitting diodes, each of said light emitting diodes having an input power rating greater than 1 and less than 10 watts and a preselected angle of tilt. Additionally, there may be a plurality of optics, each optic comprising a reflector, associated with one of said light emitting diodes and providing dispersion angles of 45-90 degrees, for enhancing light intensity. There may further be a user control area providing indicators and switches by which a user may select and confirm desired treatment parameters. The device may also include a housing substantially enclosing and retaining said light emitting diodes, optics and user control area. There may also be an optical face substantially integrated with said housing to provide a smooth surface toward the area of treatment, said optical face comprising a diffuser for uniform dispersion of light.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a block diagram of a representative control circuit for various embodiment of the present disclosure;

FIG. 2 is a perspective view of a high power LED photobiology device in accordance with one embodiment of the present disclosure;

FIG. 3 is a side view of the high power LED photobiology device;

FIG. 4 is a perspective bottom view of the high power LED photobiology device;

FIG. 5A is a plan view of the high power LED photobiology device;

FIG. 5B is a cross-sectional view along plane A-A as indicated in FIG. 5A of one variant of the high power LED photobiology device with parallel aimed LEDs and optics;

FIG. 6 is a cross-sectional side view of a LED optic assembly in accordance with various embodiments of the present disclosure;

FIG. 7 is a cross-sectional view of another variant of the high power LED photobiology device with angularly aimed LEDs and optics; and

FIG. 8 is an exploded perspective view of the operational components of the variant of the high power LED photobiology device with angularly aimed LEDs and optics as depicted in FIG. 7.

### DETAILED DESCRIPTION

The detailed description set forth below in connection with the appended drawings is intended as a description of certain embodiments of a high-powered light emitting diode (LED) photobiology device, and is not intended to represent the only forms that may be developed or utilized. The description sets forth the various functions in connection with the illustrated embodiments, but it is to be understood, however, that the same or equivalent functions may be accomplished by different embodiments that are also intended to be encompassed within the scope of the present disclosure. It is further understood that the use of relational terms such as first and second and the like are used solely to distinguish one entity from another without necessarily requiring or implying any actual such relationship or order between such entities.

The present invention generally relates to compositions, methods and systems comprising high-power light-emitting diodes (LEDs) for use in human and/or animal phototherapy applications. Phototherapy uses light of selected wavelengths to, for example, help repair damaged skin, improve health, alleviate pain, and accelerate the healing process. For example, the FDA has issued FDA Predicate Device 510(k) Clearance for: Medical Aesthetics (878.4810, GEX, OHS Wrinkles, Benign/Pigmented Lesions, PDT, Acne, etc.), K082586 (Lightwave), K062991 (GentleWave), K103415 (Tanda); Body Contouring/Cellulite (878.4810, OCI, NUV, ILY), 20 K082609 (Erchonia Zerona), K0101366 (ilipo, Utra); Androgenic Alopecia (890.5500, OAP), K122248 (igrow Hair, TheraDome); and Pain Relief (890.5500, ILY), K112494 (Varaya P C).

In some embodiments, there is provided a combination of high-powered light-emitting diodes (LEDs) each having specific properties of optical output power at specific wavelengths of emission. The LEDs may be equipped with specific integrated optics adapted to the respective wavelengths of the LEDs. The present disclosure also contemplates a phototherapy (photobiology) device with high-powered LEDs providing adjustable optical power output at predetermined wavelengths and associated methods for the beneficial application thereof. In further detail, the photobiology device has adjustable, tilted LED angles of incidence. In certain embodiments, the angle of tilt is from 1 to 45°. An improved healing of tissue, symptomatic pain relief, physical medicine/rehabilitation, and anti-aging procedures for treatments of an individual are envisioned. The treatments may be applied by a professional, and alternatively, by consumers themselves.

Predetermined ranges of light wavelengths are understood to promote wound healing and other beneficial processes contributing to anti-aging and relief from a diversity of maladies. A range of light frequencies is indicated by various colors (i.e., wavelengths) of the spectrum. Using various wavelengths, colors relatively near to one another on the light spectrum may cause different effects when applied to various portion of the body.

For example, specific wavelengths of light at specific intensities have been found to aid tissue regeneration, resolve inflammation, relieve pain, and boost the immune system. While the underlying mechanisms of phototherapy benefits are the subject of ongoing investigations, it is widely accepted that a principle mechanism is photochemical in nature, and is not heat-related.

Observed biological and physiological effects include changes in cell membrane permeability, and up-regulation and down-regulation of adenosine tri-phosphate (ATP) and nitric oxide (NO). One embodiment of the present disclosure contemplates an enclosure for protecting and arranging the components. There may be a power source and converter for use of either AC or DC power. Furthermore, there may be a cooling component configured to provide cooling of device components, as well as a heat sink component configured for effective heat transfer. The device may include controls to enable user-selective on/off operation, LED/wavelength selection, operation and/or combination, and device reset. Additionally, there may be a timing circuit for user-selected dosage periods of, for example, 1 to 5 minutes. The device may have a light emitter component with a plurality of LEDs including, but not limited to: a blue LED and associated optic providing emission at or around 415 nm (nanometers); a green LED and associated optics providing emissions at or around 525 nm; an amber LED and associated optics providing emissions at or around 590 nm; a deep red LED without optics or a deep red LED with associated optics providing emission at or around 660 nm; and an infrared LED with associated optics providing emission at or around 850 nm.

The input wattage ratings for LEDs of the present invention may be greater than 1 watt in some embodiments. Power levels of 1 watt or less may be insufficient for therapeutic non-contact use of a handheld photobiology device of the present invention. In other embodiments, the wattage ratings for LEDs of the present invention are between 1 and 10 watts. In preferred embodiments, LED input wattage ratings between 1 and 10 watts provide both contact and noncontact therapeutic treatments to be enhanced. In further embodiments, power levels do not exceed 10 watts.

In some embodiments of the present disclosure, a light emitter component of the device may comprise a combination of discrete LED devices. The LED devices may be selected and arranged to radiate light over a predetermined range of specific wavelengths or combinations of predetermined ranges of wavelengths. The LED devices and associated electronic controls and circuitry are provided in an enclosure for protection and convenient use.

The device provides a combination of high-power light-emitting diodes with specific optical output at predetermined wavelengths. Optics adapted to specific wavelengths may be provided to achieve desired direction and distribution of energy. Such optics, also known as reflectors, lenses or collimators, are configured for the intense light of a LED to be diffused or spread evenly across a broad emitting surface with reduced loss of energy intensity thereby directing the beneficial light evenly across a wide treatment area. In this fashion, the need for multiple redundant arrays of LEDs and heat generation is reduced without loss of efficiency.

Conventional LED phototherapy devices are hampered by significant loss of power at distance. For example, conventional phototherapy devices may lose more than 50% of their emitted power at a distance of ½″. On the other hand, high-power LEDs with reflector optics having dispersion angles of approximately 45-90 degrees as contemplated can deliver desired light output with uniform intensity diffused across a large area in contact or non-contact methods of treatment. Conventional phototherapy devices require skin contact with the phototherapy device in order to deliver the desired treatment. Such contact is understood to entail risks associated with microbial transfer, contamination, and patient discomfort when treating sensitive or difficult to reach areas. The devices of the present disclosure eliminate the need for direct contact by employing reflector optics and high-power LEDs having predetermined frequency outputs. Non-contact treatment further addresses treating sensitive, painful, or difficult to reach areas of the body. In turn, by incorporating the emitting surface of the optics into the surface of the housing, some embodiments of the device may be configured to be quickly and easily cleaned and sterilized between uses.

A high-powered light-emitting diode photobiology device having human/animal application in accordance with various embodiments of the present disclosure may overcome the identified shortcoming of conventional devices. In particular, the device may have a sealed light-emitting surface for enabling cleaning and sterilization of the devices prior to use. In further embodiments, the device may have optics associated with LEDs for controlling diffusion and intensity of emitted light over a larger area to improve treatment efficacy. The device may have selected combinations of predetermined light frequencies for use over a range of treatment durations.

The present disclosure further contemplates a high-powered light-emitting diode photobiology device for treatment of, and applications including, tissue repair, wound healing, and prevention of tissue death. Additional applications include relief of inflammation, pain, edema, and acute and chronic diseases. Furthermore, there may be applications including neurogenic pain, neurological problems including neuronal toxicity, nerve regeneration, and stimulation. Treatments involving traditional Chinese medicine/color-puncture, stimulation of acupuncture/trigger points (1-40 mm), and Bonghan channel hyaluronic acid/stem cells are also possible. Behavioral healthcare/psychiatric treatment including Seasonal Affective Disorder (SAD), depression, anxiety, Post-Traumatic Stress Disorder (PTSD), addiction, pain and sleep disorders alone or in combination with conventional therapeutic modalities, e.g. cognitive-behavioral, biofeedback, EMDR, deep relaxation, etc. are also possible in accordance with the presently disclosure. The device may be used in connection with the treatment of, and applications including, musculoskeletal system (muscles, ligaments, tendons, joints, bones) repair, improved strength and flexibility.

Applications including syntonic optometry (although direct viewing of light is not recommended) are also possible. The device may be utilized in the treatment of, and applications including, non-invasive trans-cranial therapies. In general, aesthetics, allergy management, athletic training, cardiology, dentistry, dermatology, disaster medicine, endocrinology, gastroenterology, general medicine, gerontology/geriatrics, gynecology, hematology, immunology, infectious disease, military medicine, neurology, obstetrics, oncology, ophthalmology, palliative medicine, psychiatry/behavioral healthcare, pulmonology, radiology, rehabilitation medicine, rheumatology, sexual health, sleep medicine, sports medicine, surgery, toxicology, urology, veterinary medicine, traditional Chinese medicine, neurogenic pain, neurological problems including but not limited to neuronal toxicity, nerve regeneration and stimulation, and syntonics are envisioned. Syntonics, (i.e., optometric phototherapy), describes a branch of ocular science that applies select light frequencies (or wavelengths) to the eyes to treat a variety of visual dysfunctions including lazy eye, and problems with focusing and convergence.

In other embodiments, the high-powered LED photobiology device or devices are contemplated for treatment in connection with aesthetics, athletic training, cardiology, dentistry, dermatology, disaster medicine, endocrinology, gastroenterology, general medicine, gerontology/geriatrics, gynecology, hematology, immunology, infectious disease, military medicine, neurology, obstetrics, oncology, ophthalmology, palliative medicine, psychiatry/behavioral healthcare, pulmonology, radiology, rehabilitation medicine, rheumatology, sexual health, sleep medicine, sports medicine, surgery, toxicology, urology, veterinary medicine, traditional Chinese medicine, and syntonics.

Compared to laser phototherapy, LEDs in accordance with the present disclosure generate non-coherent, or out-of-phase light wherein the light waves are not synchronized thereby providing a safe, diffused light source that does not burn or damage tissue. Unlike conventional laser phototherapy, the present disclosure provides continuous high-powered LEDs, having specific optical output power(s) at specific wavelengths. LED devices of the present disclosure further comprise specified optic enhancements configured to promote the efficacy of their respective wavelengths, and provide a safe diffused light source in contrast to the burning or similar damage that may occur with use of a laser.

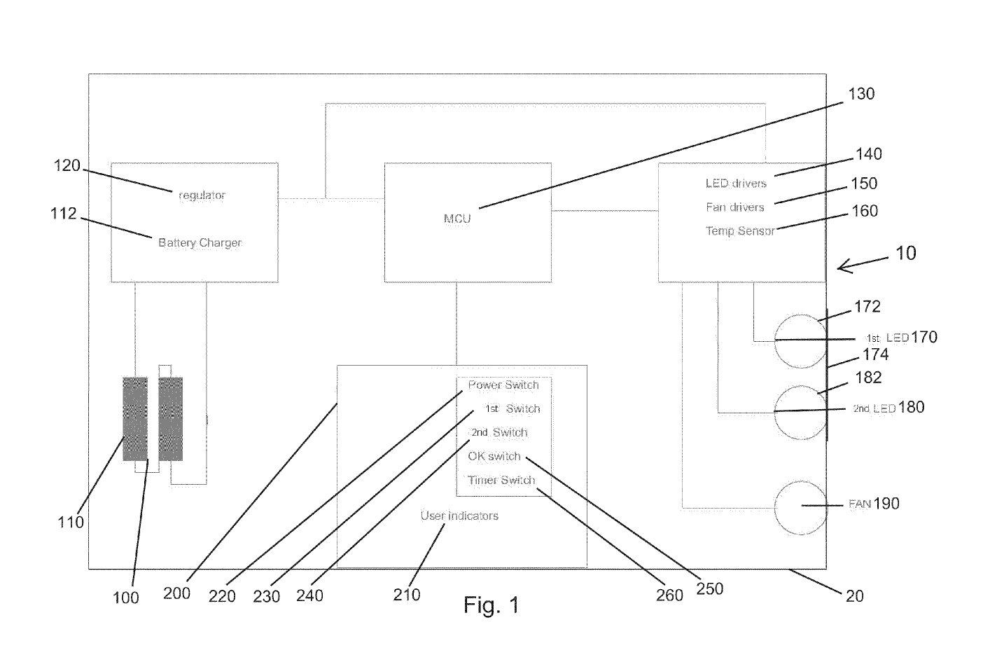

Referring now to FIG. 1, an embodiment of a high-powered LED photobiology device 10 in accordance with the present disclosure comprises a housing 20 adapted to at least partially surround the components in order to provide necessary protection and facilitate handling and manipulation by a user. First optic 172 and second optic 182 may be preferably integral to housing 20 to facilitate construction but not necessarily so. Such optics 172, 182 are preferably arranged to deliver 45-degree output angle of dispersion. Output angles for suitable optics may range from 45-90 degrees. An example of such optics is part no: RGB-1WS-LM45, Lens and Mount Assembly, available from Super Bright LEDs Inc. With efficiency as high as 90%, such optics are suitable for devices contemplated by the present disclosure. Performance achieved through the use of optics is improved through a combination of reflective and diffusive surfaces to provide the desired output angle of dispersion, and even distribution of light output across the output face.

By integrating the necessary optics in the construction of the device, the optics 172 and 182 may be combined with a housing 20 to provide a sealed surface enabling ease of cleaning and sterilization. The LEDs 170, 180 are positioned with respect to the optics 172 and 182, respectively, to provide the spatial radiation pattern desired for a chosen treatment. The degree of angular displacement of light intensity produced by the LED 170 or 180 is relative to the distance at which the device may be held with respect to the area to be treated. Optionally, a diffuser 174 is preferably employed to achieve greater uniformity of the dispersed light energy. The diffuser 174 includes a translucent or frosted layer of suitable material, often plastic or glass. Furthermore, the diffuser 174 is preferably integral with housing 20, or may be incorporated into the construction of the reflectors 172 and 182.

The LEDs 170, 180 may be selected to generate light of different frequencies. Different selected light frequencies are understood to produce different muscle contraction frequencies. By combining the two LEDs 170, 180, the device 10 creates a frequency interference pattern of muscle contraction frequencies. This interference pattern produces stimulation similar to electrical muscle stimulation products without the need for direct electrical contact with the patient. The incorporation of near-infrared or infrared frequencies enables the device 10 to achieve treatment with levels of energy penetration in marked contrast to prior art devices.

A power supply 100 is connected to micro-controller unit (MCU) 130 to enable powering of the device 10. Optionally, power supply 100 may be connected to battery charger 112, battery 110, and regulator 120 to enable the device to be used while free from an AC power cord connection. The Micro-Controller Unit (MCU) 130 is connected to the power supply 100 of choice, and the LED drivers 140, fan drivers 150 and a temperature sensor 160, each of which is also connected to power supply 100 as necessary. As used herein, the phrases “connected to” and “coupled to” to refer to any form of interaction between two or more entities, including mechanical, electrical, magnetic, electromagnetic, fluid, and thermal interaction. Two components may be coupled to each other even though they are not in direct contact with each other.

The MCU 130 receives signals from LED drivers 140, the fan drivers 150 and the temperature sensor 160 and, in accordance with software code programming well known in the art, delivers controlling signals to the LED drivers 140 to provide the light output desired. Similarly, the MCU 130 delivers controlling signals to the fan drivers 150, at least partially in response to signals received from the temperature sensor 160, to operate a fan 190 in order to prevent components of the apparatus of the present disclosure from overheating.

The LED drivers 140 are each associated with one or more of the first LED 170 and the second LED 180. Additional LEDs, not shown, are contemplated as being within the scope and breadth of the present disclosure. The first LED 170 and second LED 180 are positioned in relation to first optic 172 and second optic 182 in order to utilize said optics to apply the desired light wavelengths in a preferred direction for application.

The apparatus 10 further comprises a user control 200 that provides a location, either upon or incorporated in said housing 20, for user indicators 210. The user indicators 210 are connected to the MCU 130 to enable a user to interact with the apparatus 10, including operating the device and ascertaining the status and condition of the device relative to use. Such user indicators 210 include a power switch 220, a first switch 230 and a second switch 240. The first switch 230 and the second switch 240 are connected to the MCU 130, and together with the LED drivers 140 allow a user to indicate and obtain the pattern of LED light desired. Additional switches, not shown, are contemplated as also being within the scope and breadth of the disclosure herein. An “OK” switch 250 and a timer switch 260 are also connected to the MCU 130. The OK switch 250 enables a user to register approval for selected settings of apparatus controls and features. The timer switch 260 enables a user to select a desired duration of high-powered light generation.

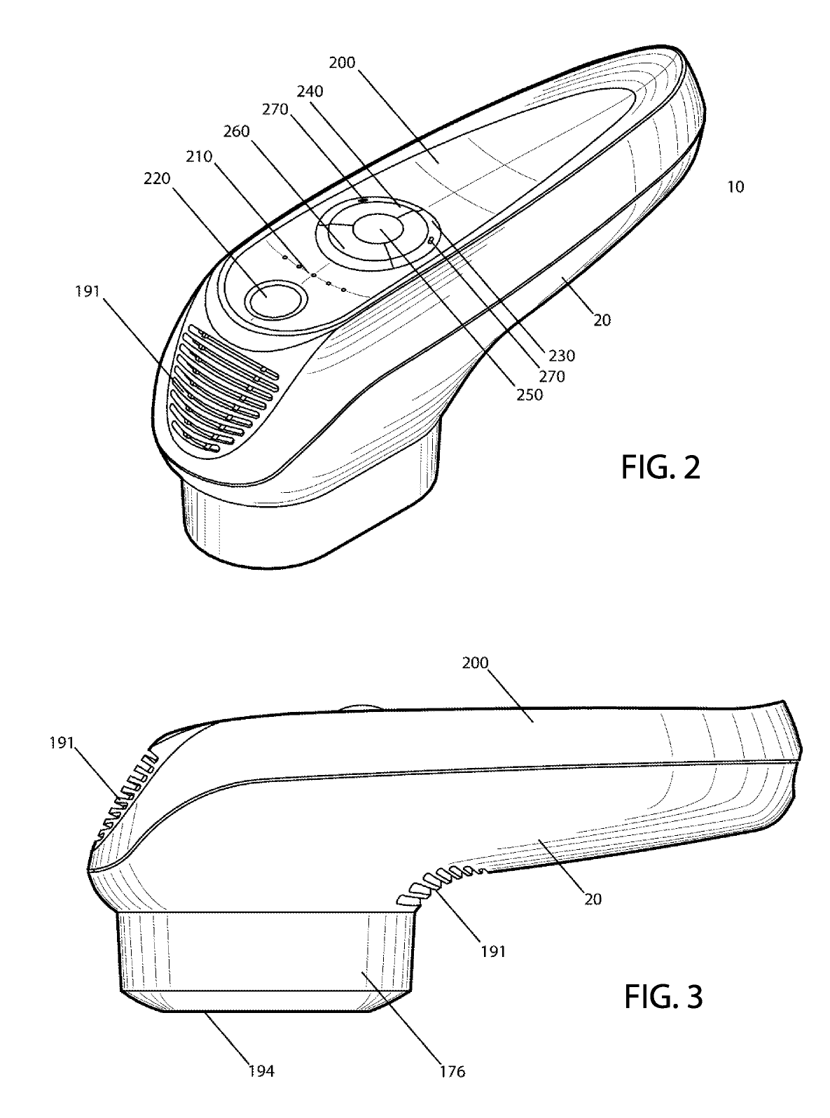

Referring now to FIGS. 2 and 3, the power supply 100 is shown as an AC power cord to provide for a corded version of the apparatus 10. The housing 20 is shown as an ergonomic and both tactilely and visually appealing form, emphasizing the hand-held size and convenient configuration of the apparatus 10.

The performance of a fan 190 is improved through the provision of depicted vents 191 formed in the housing 20 adjacent to the fan 190. A user control area 200 is provided with a layout and configuration that is practical, easy to clean and easy to use. User indicators 210 provide the user with information about the device status and control. The power switch 220 enables a user to easily turn the device 10 on and off. The timer switch 260 and the OK switch 250 are depicted in convenient and stylish arrangement with the first switch 230 and the second switch 240. A plurality of indicator lights 270 may be coupled to one or more other controls to improve feedback to a user.

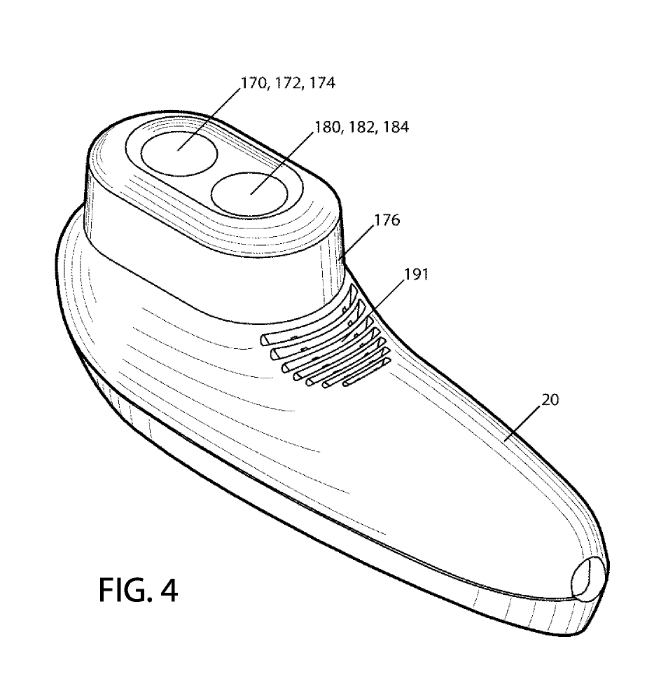

Referring now to FIG. 4, the first optic 172 and the second optic 182 are shown in cooperative arrangement with the ergonomic and elegantly functional design of housing 20. The first LED 170 and the second LED 180 are not directly visible in FIG. 4, but are indicated in their relative position centrally arranged within the first optic 172 and the second optic 182. Additional optics and associated LEDs are contemplated as being within the scope and breadth of the present disclosure.

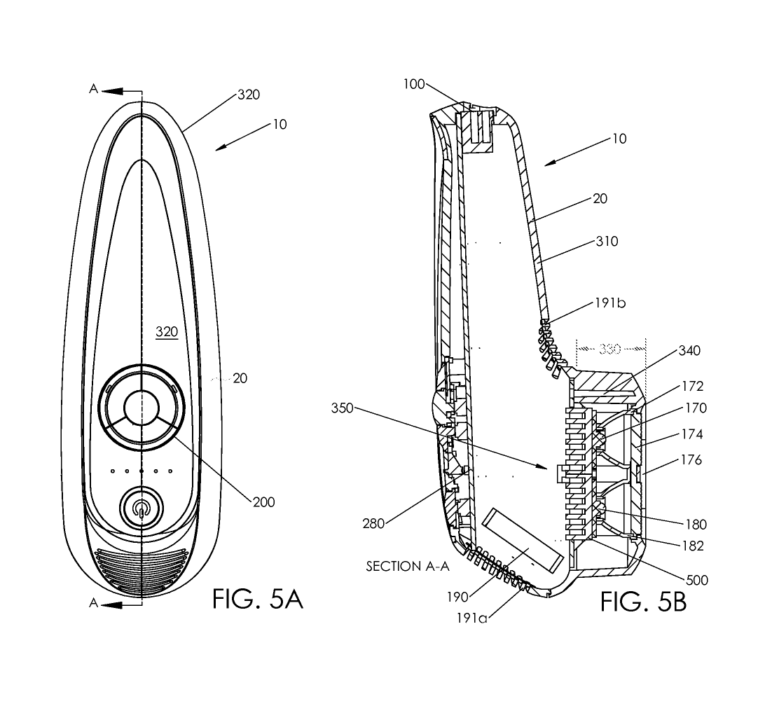

FIG. 5A and FIG. 5B depict a plan view and a side sectional view along the A-A plane, respectively, of the device 10 in accordance with one embodiment that utilizes parallel aimed optics. The power supply 100 is shown as a receptacle for a plug-in style connector to an external power source. The aforementioned electronic components, including MCU 130 and the various user controls 200 are mounted to a printed circuit board 280, which is enclosed within the housing 20. In further detail, the physical configuration of the high-powered LED photobiology device 10 is generally defined by the housing 20 having an elongate handle portion 300 that may be gripped with one hand by the user. The housing 20 is further defined by an underside 310 and an opposed top surface 320. In a typical use case, the underside 310 may rest on the index, middle, ring, and small fingers, while the thumb rests on the top surface 320. The aforementioned user controls 200 are accessible from the top surface 320 for operation by the thumbs. The housing 20 is further defined by an emission portion 330 that projects from the handle portion 330 in a generally perpendicular relationship thereto and further defined by the optical face 176, though other embodiments with angles offset from perpendicular are also contemplated. The optical face 176 provides for a sealed surface that is easy to clean and sterilize as needed, and which provides a smooth surface for skin contact when required for treatment.

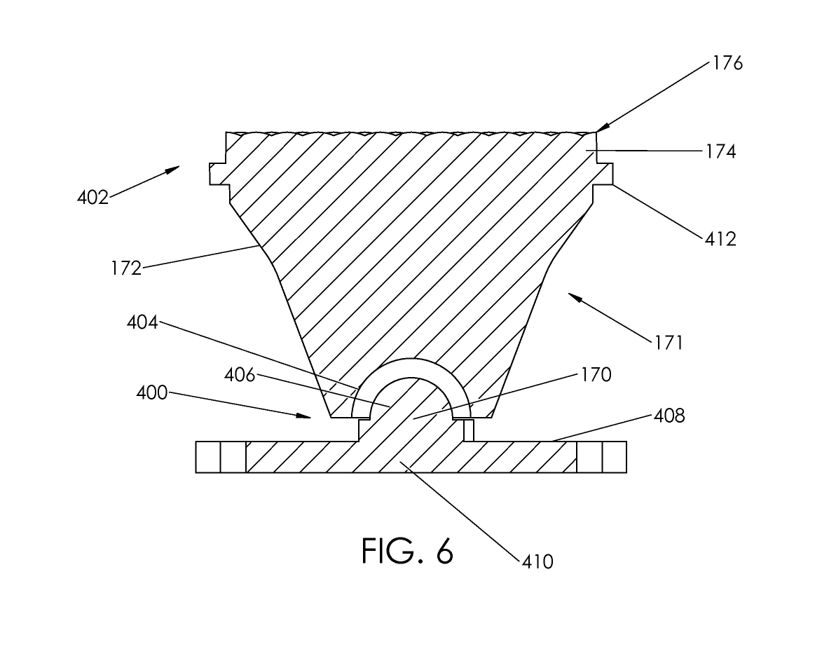

The interior of the housing 20, particularly at the emission portion 330, defines a slot 340 within which an emitter assembly 350 is disposed. The emitter assembly 350 includes the aforementioned first LED 170 and second LED 180, as well as the first optics 172 and the second optics 182. In accordance with some embodiments of the present disclosure, there may be an integral diffuser 174 that extends across both the first optics 172 and the second optics 182, though separate and discrete diffusers 174 may be provided for each. These components may be referred to collectively as a light emitting diode optics assembly 171, a cross-sectional view thereof being shown in FIG. 6. Although the details of only the first LED 170 and the first optics 172 will be provided, those having ordinary skill in the art will recognize that such details are applicable to the second LED 180 and the second optics 182.

The first LED 170 is positioned relative to optic 172 for effective collimation and dispersion of generated light energy. The optic 172 has a generally conical configuration defined by an emission source apex end 400 and an opposed, emission output base end 402. In further detail, the optic may be a smooth surfaced parabolic (45-90° preferably 45°) chromated reflector that is connected and seated on top of a light chip. In some embodiments the reflector touches the chip board and the entire surface is coated with reflective material. In other embodiments, light that escapes from the optical lens is captured and redirected toward the treatment surface by the reflector, thereby reducing light from escaping, decreasing non-coherence or divergence/diffusion, increasing spatial coherence, and decreasing loss of power intensity allowing for non-skin contact and distance/treatment choice; and 2) an acrylic, integrated, conic, pipe stem lens recessed on top of the parabolic reflector. In some embodiments, the diffusing lens extends down to meet an LED emitter and does not touch the chip board. In other embodiments, the end of optical lens is concave, and wraps around to capture more of the emitted light and light pipe it to the emitting surface with increased collimation. In certain embodiments, light that escapes the optical lens is captured and redirected toward the treatment surface by the parabolic reflector and emits through an array of lenses. In further embodiments, a lens surface has an array of small patterned domes which reduce power density loss of non-coherent light up to 20%.

The diffuser 174 is understood to be a translucent component defined by a series or a pattern of angularly offset edges that disperses and reduces the coherency of light. The diffuser 174 increases power density and efficacy of non-coherent light, and is configured to function specifically with a high-powered LED chip. The LEDs of the present disclosure provide optics that do not have a “hot spot” typical of lasers, but that provide an even energy distribution across the field of a treatment area. In preferred embodiments, LEDs with optic enhancements equal or exceed the therapeutic range (Hamblin et al, below) of “cold”, non-thermal damaging lasers but without the hazards, cost or restrictions of laser light sources.

In a typical configuration, the first LED 170 is comprised of a diode element 406 that generates light emissions upon being energized with electrical power. The diode element 406 may be mounted to a substrate plate 408 or light chip, and encapsulated within a lens 404, which may be a translucent or transparent material. This is understood to increase collimation and spatial coherence, reduce divergence, increases power density over a treatment area, and permits non-skin contact. The substrate plate 408 further includes conductive terminals 410 that are electrically connected to the respective cathode and anode of the diode element 406. The conductive terminals 410, in turn, are understood to be connected to outputs of the LED drivers 140.

Additionally referring to the cross-sectional view of FIG. 5B, the emission output base end 402 may have a flange 412 that can be engaged to a corresponding slot on the housing 20 in a locking relationship, thereby securing the optic 172 thereto. The substrate plate 408 is attached to a heat sink 500, which is understood to dissipate the heat generated by the LEDs 170, 180 and conducted to the substrate plate 408. In this regard, the substrate plate 408 is understood to be constructed of a highly thermally conductive material such as aluminum alloys, as is the heat sink 500 itself. The interface between the substrate plate 408 and the heat sink 500 may include a thermal grease or adhesive. The heat sink 500 is defined by a plurality of fins that effectively increase the surface area in contact against the surrounding air that can dissipate the heat. Furthermore, the heat sink 500 is placed within an airflow path defined between an inlet vent 191a and an outlet vent 191b. Adjacent to the inlet vent 191a is the aforementioned fan 190, and is angled such that the face of the fan 190 is parallel with the opening of the inlet vent 191a. The output face of the fan 190 is angled relative to the heat sink 500, thus forcing airflow against the fins thereof and toward the outlet vent 191b. In addition to cooling the LEDs 170, 180, this cooling system is envisioned to reduce the possibility of harmful temperature effects on the MCU 130, the LED drivers 140, and the fan drivers 150. For example, in certain embodiments, a 17° C./W design comprises an N19-20B heat sink with attached fan.

As indicated above, the embodiment of the emitter assembly 350 contemplates the emission direction of both the first LED 170 and the second LED 180 to be parallel to each other, and generally coaxial with a normal axis to the optical face 176. Accordingly, the substrate plate 408 of each can be positioned in a coplanar relation. With the two substrate plates 408 defining a single planar surface, a single heat sink 500 defining a monolithic planar surface can be utilized. The first LED 170 and the second LED 180 may be driven concurrently as described in further detail below, and because of the dispersion characteristics attributable to the first optic 172 and the second optic 182, some degree of spatial overlap in the emissions is contemplated.

One embodiment of the present disclosure is equipped with a green (525 nm) 3 watt LED with accompanying optic; an amber (590 nm) 3 watt LED with accompanying optic; a near infrared (850 nm) 3 watt LED with accompanying optic; and, a red (660 nm) 5 watt LED with or without optic. In another embodiment of the present disclosure, the device 10 is equipped with a green (525 nm) 1 watt LED with accompanying optic; an amber (590 nm) 1 watt LED with accompanying optic; a near infrared (850 nm) 1 watt LED with accompanying optic; and, a deep-red (660 nm) 5 watt LED with or without accompanying optic. In yet another embodiment of the present disclosure, a device is equipped with an amber (590 nm) 3 watt LED with accompanying optic; and a red (660 nm) 5 watt LED with or without accompanying optic. In another particular embodiment of the present disclosure, the device 10 is equipped with a deep red (660 nm) 5 watt LED with or without optic; and, an infrared (850 nm) 3 watt LED with accompanying optic. In a further embodiment of the present disclosure, the device 10 is equipped with a 415 nm LED and a red (660 nm) LED, each LED being rated from 1 to 10 watts and used with or without accompanying optics.

All wavelengths and/or ranges of wavelengths set forth herein are understood to be within a range of plus or minus 5 nanometers. All of the optics disclosed herein provides beam angles of distribution within ranges of plus or minus 5 degrees of the given value/s.

In some embodiments, the present disclosure provides for the application of light for a selectable period of time, generally from 1 to 5 minutes, with the light directed at and in relatively close proximity (generally, from 1 to 4 inches) to the area for treatment. Such treatment methods also comprise a repetition of applications of device light at a frequency of from one or more times a day.

In some embodiments, for treatment of wounds and/or healing of superficially bruised tissue, a high-powered red LED having an emitted light frequency of 660 nm is provided as one of the LEDs 170 and 180 of the device. Using user control 200, a user selects the high-powered red LED from switches 230 and 240, and selects a desired treatment time with timer switch 260. The settings are confirmed with the OK switch 250 to initiate a treatment session. The user then places the optical face 176 in proximity to the wound or bruised tissue to be treated. The device 10 may be oriented with the optical face 176 stationary and parallel to the skin surface area to be treated. In accordance with one embodiment, for wounds and bruised tissue, the optical face 176 is to be positioned within two inches of the treatment area. Treatment duration may vary from 30 seconds to 5 minutes per area, and may be repeated daily as needed.

In some embodiments, for treatment of pain and/or superficial muscle strain, a high-powered red LED having an emitted light frequency of 660 nm is utilized for one of the LEDs 170 and 180. A user selects the high-powered red LED and employs a treatment with the addition of therapeutic gentle stretching of the tissue/muscle in and away from the optical face 176. To treat deeper tissue or strained muscle, a high-powered near infrared LED having an emitted light frequency of 850 nm is provided as one of LEDs 170 and 180. For superficial penetration, myofascial trigger points, and for relief of neuropathic pain, red LED output is applied for between 1-5 minutes at a distance of from 2″ to contact with the treatment area. Such treatment is repeated from daily to three times per week as needed. For deeper penetration and trigger point stimulation and joint injuries, the near infrared LED output is applied for between 2 to 6 minutes. Such treatment is repeated from daily to three times per week as needed.

In some embodiments, for facial toning and/or photo rejuvenation, a high-powered amber LED having an emitted light frequency of 590 nm is provided as one of LEDs 170 and 180. The user smiles gently while applying light treatment once every other day for 1 to 5 minutes. Light application covers the area under the chin, the entire face and top of scalp, and behind the ears. The mouth should be opened during treatment while smiling, and light should be applied to the inside of mouth and cheek muscles. Direct application of light to the thyroid gland, however, should be avoided. To stimulate acupuncture or trigger points, light from the amber LED should be applied to selected points once a day for a time period of from 30 seconds to 3 minutes. Total time of light application should not exceed 5 minutes.

Phototherapy devices comprising, for example, super luminous light diodes (SLDs) or LEDs may provide treatment either through photo-thermal, tissue destroying processes (i.e., “photo-thermolysis”), or photo-chemical, non-thermal processes (i.e., “low level light therapy (LLLT)”, “photobiology (PB)”, “photobiomodulation”, “biostimulation/bioinhibition”) or, as used herein, “PB/LLLT”. In some embodiments, PB/LLLT is not a thermal or tissue destroying process unlike high energy density laser procedures, but is a photochemical process. In preferred embodiments, PB/LLLT power density is lower than that needed to heat or destroy tissue, for example, less than or equal to 100 mW/cm². In certain embodiments, a discrete light source optical output level is less than or equal to 500 mW. In some embodiments, PB/LLLT comprises a biphasic dose response compatible with the “Arndt-Schulz Law”, a model that describes dose dependent effects of PB/LLLT and consequent cellular biostimulation and bioinhibition. Biostimulation standards typically fall within 1-7 J/cm² and bioinhibition standards are 10 J/cm² or more and should not exceed 100 J/cm². Accordingly, power density (irradiance) and energy density (fluence) may, in some embodiments, define an optimal therapeutic window. In turn, target power density and size of treatment area may also indicate energy density and delivery time. (See, for example, Hamblin M, et al. Biphasic Dose Response in Low Level Light Therapy—An Update. Dose Response. 2011; 9 (4): 602-618.)

In some embodiments of the present disclosure, the high power LEDs provide a light source that overcomes many disadvantages of laser light. Laser light is typically coherent, collimated, and provided with a narrow beam with little or no divergence. For example, laser light divergence may be under 1° and hazardous to the eye. If provided as a continuous wave laser light results in bulk overheating and nonselective tissue damage. Pulsed laser light allows tissue to cool between the pulses. With laser treatment, target sizes must typically be small (e.g., 0.25-0.5 cm²). Moreover, laser light treatment typically requires skin contact to avoid eye damage. To achieve maximum penetration, laser light must often be applied in a grid, or with overlapping treatment areas. In turn, laser light sources are more expensive than SLDs or LEDs. Current controversy exists whether the treatment application time of lasers produces optimal photobiological responses in view of a correlation between power/energy density and time. Cited Source: Allemann I B, and Kaufman J, 2011. Laser Principles. Bogdan Allemann, Goldberg, D J (eds.): Basics in Dermatological Laser Applications. Curr. Probl Dermatol, Basel, Karger, vol 42, pp 7-23.

SLDs typically provided in a t-pack assembly often deliver insufficiently uniform lighting, are not heat-sinked, and are bulky in size due to the dimensions of each t-pack. Conventional SLD t-packs are low in discrete power, are not heat providing, and produce highly divergent light. A further disadvantage of SLD light sources for therapeutic, cosmetic and other applications described herein is that SLD-based devices require skin contact to overcome the high light divergence, and the non-coherence of the light source. In turn, SLDs require longer treatment intervals to overcome low energy density. Accordingly, SLD t-packs may not cannot provide required performance. Compared to lasers and SLDS in conventional PB applications, high powered chip-on-board LEDs of the present disclosure provide the required performance, do not deliver energy sufficient to cause thermal damage, and do not share the risk of accidental eye damage as laser light sources. (See, for example, Barolet D, M. D.: 2008. Light Emitting Diodes (LEDs) in Dermatology. Sem. Cutaneous and Medicine and Surgery 27:227-238.)

LEDs without optic enhancements decrease intensity or energy density with distance from the skin or other surface. In some embodiments, the present disclosure provides LEDs with optic enhancements that reduce loss of energy density at a distance from a surface that is otherwise inherent to LEDs without optic enhancements. Accordingly, in some embodiments, LEDs of the may emit light that is non-coherent, are divergent or non-collimated, and decrease intensity with increasing distance from skin contact. In certain embodiments, LEDs radiate a non-coherent cone up to 60° from centerline. In other embodiments, high-powered LEDs are provided as chips, with each discrete chip configured in dimensions that allow multiple wavelengths with high power density output in a small area. For example, a discrete LED with optic enhancements requires 10 cm² of surface with an individual input power of >1 W and <10 W. In other embodiments, a discrete LED chip is wavelength specific and approximately 10 cm² in space required. In some embodiments, a discrete LED chip is wavelength specific and approximately 10 cm² in space required. In other embodiments, depending on input power, a discrete LED is capable of replacing numerous, bulky SLD t-packs or matching more expensive laser energy density and treatment times without risk of thermal injury. In preferred embodiments, LEDs are easier to apply than a laser (e.g., grid pattern), and cover a larger treatment area size in a single application.

In particular embodiments of the present disclosure, a high powered LED is provided with input power between 1 W and 10 W 500 mW/cm² for the contemplated PB/LLLT applications. In a preferred embodiment, continuous wave high powered LED of 3-6W input power produces, for example, 25-350 mW of optical output power, a power density of 8-30 mW/cm² for Green/Blue/Amber, and 25-100 mW/cm² for Visible Red/near Infrared wavelengths, and a treatment size area of 10 cm² at 0.5″ from skin to 76 cm2 at 2″ from skin as shown in Tables 1, 2A-C, and 3 below. These parameters are within photobiological parameters of less than 500 mW or 100 mW/cm² and meet or exceed minimum statistical significance of 4 mW/cm² (Green, Amber) and 25 mW/cm². (Red, IFR). Hamblin M, et al. Biphasic Dose Response in Low Level Light Therapy—An Update. Dose Response. 2011; 9 (4): 602-618.

TABLE 1

| | Red (660) | | nIR (850) | | Amber (590) | |
|---|---|---|---|---|---|---|
| | power | | power | | power | |
| | density | energy in 1 | density | energy in 1 | density | energy in 1 |
| distance | (W/cm²) | min (J/cm²) | (W/cm²) | min (J/cm²) | (W/cm²) | min (J/cm²) |
| Optic only | | | | | | |
| 0.75″ | 0.07099 | 4.26 | 0.04985 | 2.99 | 0.02399 | 1.44 |
| 1.75″ | 0.02205 | 1.32 | 0.01376 | 0.83 | 0.00767 | 0.46 |
| 2.75″ | 0.0157 | 0.94 | 0.00995 | 0.6 | 0.00258 | 0.16 |
| 4.75″ | 0.00674 | 0.4 | 0.00377 | 0.23 | 0.00116 | 0.07 |
| 6.75″ | 0.00373 | 0.22 | 0.00194 | 0.12 | 0.00082 | 0.05 |
| 8.75″ | 0.00213 | 0.13 | 0.00119 | 0.07 | 0.00062 | 0.04 |
| Reflector | | | | | | |
| 0″ | 0.0776 | 4.66 | 0.05291 | 3.17 | 0.01411 | 0.84656 |
| 1″ | 0.04409 | 2.65 | 0.02469 | 1.48 | 0.00776 | 0.46561 |
| 2″ | 0.02963 | 1.78 | 0.02081 | 1.25 | 0.00494 | 0.2963 |

TABLE 2A

| Reflector | Red (660) | | |
|---|---|---|---|
| & Optic | | power density | energy in 1 min |
| distance | Raw Measure (mW) | (W/cm²) | (J/cm²) |
| 0-0.5″ | 304 | 0.107231041 | 6.433862434 |
| 1″ | 225 | 0.079365079 | 4.761904762 |
| 2″ | 100 | 0.035273369 | 2.116402116 |

TABLE 2B

| Reflector | nIR (850) | | |
|---|---|---|---|
| & Optic | | power density | energy in 1 min |
| distance | Raw Measure (mW) | (W/cm²) | (J/cm²) |
| 0-0.5″ | 203 | 0.071604938 | 4.2962963 |
| 1″ | 150 | 0.052910053 | 3.17460317 |
| 2″ | 71 | 0.025044092 | 1.5026455 |

TABLE 2C

| Reflector | Amber (590) | | |
|---|---|---|---|
| & Optic | Raw | power density | energy in 1 min |
| Raw Measure (mW) | Measure (mW) | (W/cm²) | (J/cm²) |
| 0-0.5″ | 54 | 0.019047619 | 1.14285714 |
| 1″ | 40 | 0.014109347 | 0.84656085 |
| 2″ | 22 | 0.007760141 | 0.46560847 |

TABLE 3

| | | Raw | | Energy |
|---|---|---|---|---|
| Bone | | Measure | Power Density | in 1 min |
| Penetration | | (mW) | (W/cm²) | (J/cm²) |
| Skull Front (1 | RED (660) | 13.73 | 0.00484 | 0.2904 |
| layer Seran) | nIR (850) | 12.18 | 0.0043 | 0.258 |
| Temple (1 layer | RED (660) | 13.73 | 0.00484 | 0.2904 |
| Seran) | nIR (850) | 3.48 | 0.00123 | 0.0738 |
| Jaw (1 layer | RED (660) | 3.43 | 0.00121 | 0.0726 |
| Seran) | nIR (850) | 1.74 | 0.00061 | 0.0366 |

In some embodiments, at a treatment distance from skin 0-2″, total combined input power of LEDs does not exceed 9 W for Red and nIFR. When 9 W is exceeded, tissue temperature may exceed FDA-approved thresholds and cause thermal damage. In other embodiments, the present invention provides a device reaching required temperatures within 30 seconds to 2 minutes dependent on wavelength/power choices. Unlike Red and nIFR, Blue, Green and Amber wavelengths do not raise tissue temperature over 40° C. to cause heat sensitivity reaction.

In the course of development of the present invention, it was discovered that LEDs without optics provide loss of output power at a distance from skin contact. For example, conventional LEDs lose up to 60% of their power density when moved 0.25″ from skin contact. In some embodiments of the present invention comprising collateral optic instruments (e.g., diffusers, lenses, canting), 40-50% of output power loss is retained thereby providing an apparatus capable of efficacious photobiological treatments over short treatment intervals without skin contact.

Skin contact free treatment is desired to reduce contamination, and to generate a beam of light contoured to the skin surface area. Compared to laser or SLD PB units wherein a faceplate and/or skin contact determines treatment size and dimension, in certain embodiments of the present disclosure beams of light follow the contours of the skin surface area with no skin contact.

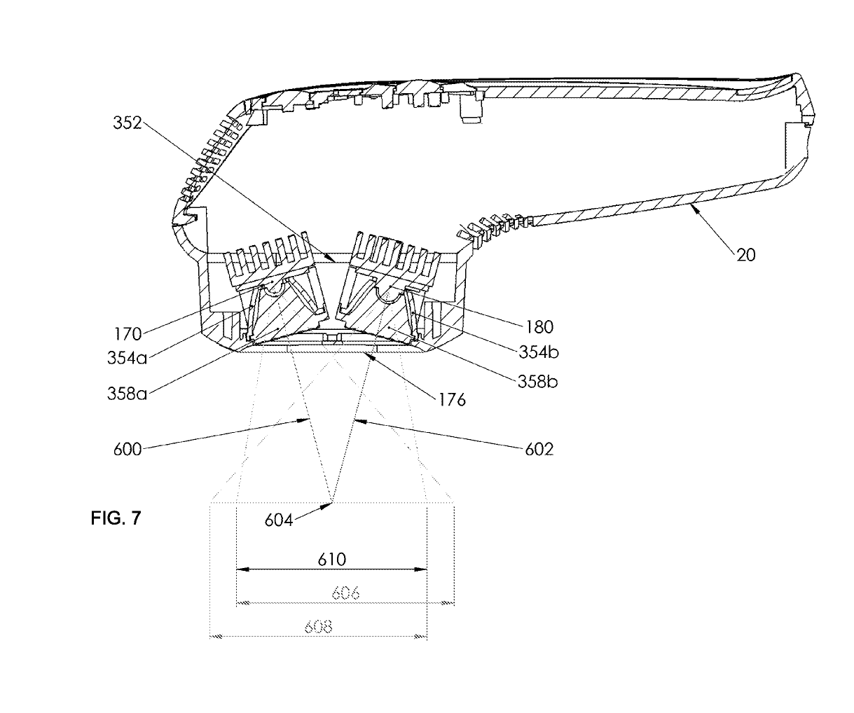

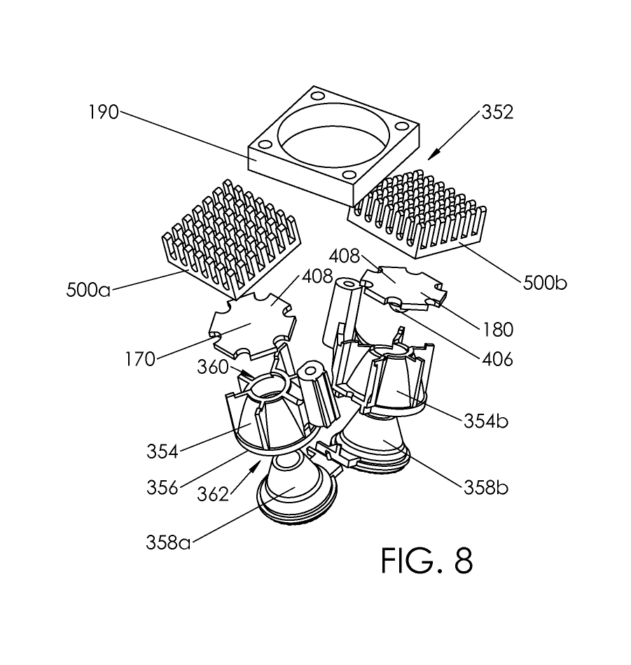

In the embodiment shown in FIG. 5B, the optical face 176 is understood to be generally perpendicular to the emission axis of both the first LED 170 and the second LED 180. With reference to FIG. 7 and FIG. 8 and the alternative embodiment depicted therein, a first emission axis 600 of the first LED 170 and a second emission axis 602 of the second LED 180 are contemplated to be offset from normal relative to the optical face 176. In other words, the first LED 170 and the second LED 180 are angled/tilted and not parallel to each other. This embodiment likewise includes the housing 20 with the emission portion 330 extending therefrom, but an alternative configuration of an emission assembly 352 is utilized.

More particularly, the emission assembly 352 includes a first tilted lens housing 354a and a second tilted lens housing 354b. The tilted lens housings 354 are defined by a socket 356 receptive to a conical lens 358, which is understood to correspond to the aforementioned optics 172, 182 of the first embodiment. There is a first conical lens 358a received within the socket 356 of the first tilted lens housing 354a, and a second conical lens 358b received within the socket 356 of the second tilted lens housing 354b. The conical lens 358 may be secured to the tilted lens housing 354 in a variety of ways, including frictional retention, threaded engagement, by adhesive compounds, and so on. Both tilted lens housings 354 have an open apex end 360 and an opposed open base end 362. The open apex end 360 receives the LED 170, which, as described above, includes the diode element 406 and the substrate 408. There are separate heat sinks 500a, 500b, corresponding to the first LED 170 and the second LED 180, respectively.

In order to maintain each of the elements of the emission assembly 352 in the angular position depicted, the housing 20 is understood to include specifically angled structures against which the tilted lens housings 354 are positioned. Again, as illustrated in FIG. 7, the respective angular offsets of the first LED 170 and the second LED 180 are understood to result in an intersecting first emission axis 600 and a second emission axis 602 at a predefined point 604 that is vertically offset by a predefined distance from the optical face 176. Furthermore, because of the dispersion effects achieved with the optics configuration, the first LED 170 has a defined first emission pattern 606, and the second LED 180 has a defined second emission pattern 608. Overlap of the first emission pattern 606 and the second emission 608 (at an overlap region 610) is possible because of the angular offsets of the first emission axis 600 and the second emission axis 602.

With different wavelengths, emission powers, and other operational characteristics of the LEDs 170, 180, various synergistic effects beyond that which are possible with single emissions are envisioned. These synergistic biochemical effects are contemplated as part of interferential therapy, and the proven clinical results of applying two wavelengths to a target tissue area. In some cases, one wavelength may be able to penetrate a greater depth than would otherwise be possible because of the concurrent application of a second wavelength. Additionally, it is possible to see the specific areas undergoing treatment. The angled LEDs 170, 180 by definition increases the vertical distance of the light emission point relative to the optical face 176 and the convergence point 604 of the first emission axis 600 and the second emission axis 602. This allows the device 10 to be positioned closer to the target tissue area allowing for greater treatment accuracy.

Wavelength, Penetration and Tissue Temperature

In some embodiments, specific light wavelengths and combinations of wavelengths are provided for a range of conditions. An apparatus in accordance with the present disclosure provides multiple wavelengths light to small and large areas in a handheld device. In one embodiment, the present invention provides a Medical Aesthetic/Dermatology model with, for example, LED wavelength combinations of: Blue 492 nm (acne bacteria), Green 525 nm (cellulite, facial contouring), Amber 590 nm (photoaging, eczema, rosacea, facial contouring, reduced scar formation), Red 660 nm (facial/body contouring, wound healing, psoriasis, dermatitis, acne inflammation, photoaging, Photodynamic Therapy, pain/inflammation relief, alopecia), and nIFR 850 nm (facial/body contouring, photoaging, Photodynamic Therapy, pain/inflammation relief, alopecia). In certain embodiments, specific conditions may require specific combination wavelengths for treatment. For example, acne vulgaris treatment may require Blue 492 nm (bacteria) and Red 660 nm (inflammation) wavelength treatment.

Conventional PB devices typically target for Fitzpatrick skin types I-III or lighter skin pigmentation populations. The Fitzpatrick Scale (also “Fitzpatrick skin typing test” or“Fitzpatrick phototyping scale”) is a numerical classification scheme for comparison of skin

pigmentation developed in 1975 by Thomas B. Fitzpatrick, a Harvard dermatologist, as a way to classify the response of different types of skin to UV light. (Fitzpatrick, T. B. (1975). “Soleil et peau” [Sun and skin]. Journal de Medecine Esthetique (in French) (2): 33-34.) More recently it was updated to also contain non-white skin types. (Pathak, M. A.; Jimbow, K.; Szabo, G.; Fitzpatrick, T. B. (1976). “Sunlight and melanin pigmentation”. In Smith, K. C. (ed.): Photochemical and photobiological reviews, Plenum Press, New York, 1976: 211-239.; Fitzpatrick, T. B. (1986). “Ultraviolet-induced pigmentary changes: Benefits and hazards”, Therapeutic Photomedicine, Karger, vol. 15 of “Current Problems in Dermatology”, 1986: 25-68.) The scale is a recognized tool for dermatologic research into the color of skin. It measures several components: genetic disposition, reaction to sun exposure, and tanning habits:

The Fitzpatrick Scale is understood to be as follows:

Type I (scores 0-7) Light, pale white. Always burns, never tans.

Type II (scores 8-16) White; fair. Usually burns, tans with difficulty.

Type III (scores 17-24) Medium, white to olive. Sometimes mild burn, gradually tans to olive.

Type IV (scores 25-30) Olive, moderate brown. Rarely burns, tans with ease to a moderate brown.

Type V (scores over 30) Brown, dark brown. Very rarely burns, tans very easily.

Type VI Black, very dark brown to black. Never burns, tans very easily, deeply pigmented.

For example, red light from a 1-10 watt high power LED, at approximately 670 nanometers, may prevent hair loss and re-grow new hair, as well as to cause increased melanin production and protein synthesis. Although the mechanism of action is not yet fully understood, it is believed that the beneficial effects set forth herein are derived from a relatively small band of light wavelengths extending on either side of the referenced wavelengths. For this reason, light wavelengths or ranges of wavelengths herein are deemed to indicate a wavelength value having a range of plus or minus five nanometers (+/−5 nm).

Red, infrared, and near infrared light have also been used to increase collagen production and to reduce redness, dilated capillaries, and damage to the skin, as well as the reduction of fine wrinkles. They also provide symptomatic pain relief and stimulate wound healing.

Blue light has been found to reduce acne and when combined with red light, eliminates acne and reduces the scarring often associated with acne treatments.

Yellow and amber lights have been found to reduce fine lines, wrinkles, rosacea, eczema, and can help to repair sun-damaged skin.

Green light has been shown to reduce and eliminate sun and age spots, Seasonal Affective Disorder (SAD) and other psychological disorders, lighten freckles and also help promote more luminous skin condition and overall radiance of the skin.

In some embodiments of the present invention, persons with skin types I-III may wish to treat wrinkles, whereas persons with skin types IV-V may be concerned about skin/muscle toning, acne, keratosis, hyperpigmentation or dark spots. Due to their heating properties, Red and nIFR light sources may increase hyperpigmentation, darkening of skin spots, keloids, and scar formation for skin types IV-V. Accordingly, some embodiments of the present invention provide Fitzpatrick skin types IV-V with non-Red/nIFR spectrum light that lack heating properties. In other embodiments, the present invention provides devices that meet the needs of specific skin types, for example, Amber (590 nm) as a favorable wavelength for Asian or darker skin types (IV-V).

In some embodiments, the device 10 may be configured to provide long wavelengths with deeper penetration. (See, for example, Hudson D E, et al. comparing penetration of 808 nm and 980 nm light projected through bovine tissue 18-95 mm thick. 808 nm light penetrates up to 54% deeper than t980 nm light in bovine tissue. Photomed Laser Surg. 2013 April:31(4):163-8.) Similarly, Jaqdeo J R et al., report that nFIR light penetrates formalin fixed soft tissue, bone and brain in correlation with direct action of nIFR light on neural tissue. PLoS ONE. 2012; 7(10):e47460.) In some embodiments of the present invention, increasing LED output power with optic enhancements increases energy density, wherein penetration of a shorter wavelength light may be increased to be equal or greater than that of a longer wavelength light at the same site. For example, testing 1) the human skull front, 5W Red 660 nm LED (energy density/minute 0.30 J/cm²), nIFR 850 nm LED (0.26 J/cm²), and 2) the skull temple, 5 W RED 660 nm (0.30 J/cm²), nIFR 850 nm (0.07 J/cm²). (Table 3. above)

In some embodiments, tissue temperature is raised to less than 40°-45° C. for 10-15 minutes during exposure in compliance with regulatory (e.g., FDA) skin reactivity and thermal injury standards. For FDA regulatory claim and clearance of increased blood circulation and oxygenation, Listed treatment times must include the lead-in time. It is understood that there are devices reaching required temperatures within 30 seconds to 2 minutes dependent on wavelength/power choices.

In some embodiments, a treatment distance from skin of 0-2″ is preferred, with a total combined LED input power not to exceed 9 W. When 9 W is exceeded, tissue temperature exceeds FDA compliance thresholds and can cause thermal damage. In some embodiments, the present invention provides wavelengths of Blue 415 nm, Green 525 m and Amber 590 nm light for heat-sensitive skin or conditions (e.g., eczema, rosacea) instead of Red/nIFR spectrum light. Unlike Red and nIFR, Blue, Green and Amber wavelengths do not raise skin tissue temperature over 40° C., or cause heat sensitivity reactions. In some embodiments of the present disclosure, Red and nIFR light wavelengths are provided wherein raised tissue temperature is preferred (e.g., psoriasis, pain/inflammation).

In some embodiments, multiple PB/LLT dosing options are provided, comprising caregiver and user selection of light wavelengths alone and in combination, intensity of treatment, duration of treatment, site of treatment, distance of a light source from the treatment surface, incident angle of light exposure, treatment regimen (i.e., number of treatments per unit time), and use of PB/LLLT with other conventional, clinical, surgical, complementary and other treatments, and the like. For example, in some embodiments, the present disclosure provides 1-2/day treatment interval, over days 1-5 (acute), and 1-10 days (chronic), followed by as needed treatments as a treatment regimen. In some embodiments, the present disclosure provides a handheld PB/LLLT device that may be used at home thereby reducing the need for hospital, office, or professional application.

Industry parameters for PB/LLLT biostimulation and bioinhibition are generated by the World 5 Assoc. for Laser Therapy (WALT) (WALT: Sherwood House, Field Lane, Wroot, Doncaster, Oldham, DN9 2BN.), and North America Assoc. for Light Therapy (NAALT) (naalt.org). In some embodiments, the device 10 meets or exceeds regulatory guidelines and WALT/NAALT treatment protocols. In certain embodiments, the present disclosure provides a device that reaches energy density for biostimulation within 1 minute or more, and bioinhibition within 2 minutes or more, depending upon distance of the light source from the skin and its wavelength. In other embodiments, the present disclosure comprises a device that provides varying energy density options in PB/LLLT research protocols by, for example, altering distance from skin.

Transdermal Applications

Conventional PB/LLLT devices often require skin contact, with restricted treatment size, low powered, a single wavelength, and are preprogrammed or limited energy density options. Embodiments of the present disclosure overcome these limitations.

Tissue Biostimulation Including Wound Healing, Skin or Dermatology, Wound Biofilms, Bone/Neural Repair/Regeneration and Combination/Photodynamic Therapy (PDT) Applications

In some embodiments, PB/LLLT may be provided for use alone, or in combination with other therapies including as an activator in PhotoDynamic Therapy (PDT), with combination therapy (i.e., with drugs), and as an adjunct therapy for chronic wound biofilms.

A handheld PB/LLLT device is contemplated wherein tissue biostimulation can be reached within one minute depending on distance of the light source from the treatment surface. In some embodiments, the present disclosure provides light wavelength(s) options based upon Fitzpatrick skin types and conditions to be treated. In other embodiments, a device of the present disclosure is configured to access and treat difficult areas due to no skin contact and unique beam properties. In specific embodiments, the present disclosure provides a device with Green/Blue/Amber/Red/nIFR LEDs configured for tissue biostimulation in the treatment of, for example, wounds/biofilms, bone/neural repair/regeneration, combination and PDT. In certain embodiments, the present disclosure provides a device with Red LEDs for virus related diagnoses (e.g., shingles, herpes), psoriasis, and conditions known to delay the healing process. In preferred embodiments, the present disclosure provides a device with reduced cross-contamination due to non-skin contact. (See, for example: Whelan H, et al., “The NASA Light-Emitting Diode Medical Programs—Progress in Space Flight and Terrestrial Applications.” CP504, Space Technology and Applications International Forum—2000.; Yun J S, et al. describing Asian skin types (Fitzpatrick skin types IV-V) prone to excessive scar or keloid formation from post-operative wound healing reporting that Amber light application may be used safely and efficiently on Asian skin to prevent scar formation on postoperative wound healing. Dermatol Surg 2011 December; 37(12):1747-53. PMID: 21883646; Fushimi T, et al., concluding that Green LEDs promote wound healing by inducing migratory and proliferative mediators, indicating that not only Red LEDs but also Green LEDs can be a tool for wound healing. Wound Repair Regen. 2012 March-April; 20(2):226-35; Mamalis A D, et al., concluding that light-based treatment achieves favorable outcomes and a new way to manage keloids, including reducing keloid recurrence. J Eur Acad Dermatol Venereol. 2013 Aug. 27; Grendel T, et al., concluding that LLLT is able to reduce granulation tissue formation and simultaneously increase new cartilage development on tracheal incisions. Photomed Laser Surg. 2011 September; 29(9):613-8; Shen C C, et al., concluding that LLLT (660 nm, 50 mW) can accelerate the repair and regeneration of a 15 mm transected peripheral nerve in rats J Biomed Mater Res A. 2013 February; Chaves M E, et al., concluding that LED phototherapy is an effective tool in healing of breastfeeding nipple trauma. Photomed Laser Surg. 2012 March; 30(3):172-8; De Carvalho R R, et al., concluding that phototherapy is effective in prevention and reduction of severity of labial manifestations of herpes labialis virus. Patients experienced a significant decrease in dimension of lesions and inflammatory edema. Lasers Med Sci. 2010 May; 25(3):397-402; Munoz Sanchez P J, et al., concluding that LLLT (670 nm) 2 J/cm² per blister prodromal stage and 4.8 J/cm² crust or secondarily infected stages on herpes simplex type 1 is an effective treatment for initial healing and length of recurrence periods. Photomed Laser Surg. 2012n January; 30(1):37-40; Garcia V G, et al., concluding that LLLT (670 nm, 5.57 J/cm²) acts as a biostimulatory coadjuvant agent balancing the undesirable effects of nicotine on wound tissue healing. Lasers Med Sci. 2012 March; 27(2):437-43; Kim C H, et al., conclude LED phototherapy is an effective treatment of inflammatory skin disorders. Combination therapy of 850 nm and low dose FK-506 shows a significant reduction of skin lesions. J Dermatol Sci. 2013 Jun. 12; Joni G. concluding that PDT is an efficient alternative treatment for microbial infections, in particular the Red spectrum with light-absorbing photosensitizers. J Environ Pathol Toxicol Oncol. 2006; 25 (1-2):505-19; Xavier M, et al., concluding that LED therapy (880 nm, 7.5 J/cm²) effectively increases mRNA expression and IL-10 and type I and III collagen on Achilles tendon injuries. Lasers Med Sci. 2013 Feb. 13; and Demidova T, et al., “Low Level Light Stimulated Excisional Wound Healing in Mice.” Lasers in Surgery and Medicine, 39:706-715 (2007).)

Pain and Inflammation Relief Including Neurogenic/Neuropathic Pain and Rehabilitation Application

PB/LLLT for relief of conditions associated with pain and inflammation is not contraindicated over implants, the spine or head area, requires no gel or surface contact unlike ultrasound or electrical stimulation, and may be more effective than electrical treatment, including transcutaneous electrical nerve stimulation (TENS), for peripheral or central neurogenic pain. In some embodiments, there is a handheld PB/LLLT device with Red/nIFR LEDs for pain and inflammation relief. In some embodiments, the device comprises Red LEDs having an analgesic effect similar to corticosteroids. In other embodiments, the present disclosure provides a device comprising Red/nIFR LEDs for the treatment of neuropathic/neurogenic pain, including “phantom limb” pain. In further embodiments, the present disclosure provides a device comprising Red/nIFR LEDs for the treatment of acute and chronic inflammatory disorders. In some embodiments, a device of the present disclosure comprising Red/nIFR LEDs decreases use of analgesics or opiates. In further embodiments, the present disclosure provides a device comprising Red/nIFR LEDs as a treatment for chronic sensorimotor disorders (ex. Restless Leg Syndrome). (See, for example: Cidral-Filho F J, et al., concluding that Light Emitting Diode therapy (nIFR, 9 J/cm²) provides significant results through 1) activation of peripheral opioid receptors and; 2) activation of the L-arginine/NO pathway for post-operative pain. Lasers Med Sci. 2013 Jul. 6.; Kedzierski T, et al., concluding that LLLT provides significant pain reduction in patients with knee joint degenerative disease with a suprerior analgesic effect compared top TENS. Ortop Traumatol Rehabil. 2012 November-December; 14(6):537-44.; Ribas E S, et al., concluding that LLLT, after 9 sessions, probides a decrease in the intensity of debilitating stump pain from amputation surgery and increased ability to perform daily living activities. Int J Gen Med. 2012; 5:739-42.; Hanfy Hala, et al., concluding that LLLT (635-670 nm, 3-6 J/cm² on painful points) is more effective than medication for chronic pelvic inflammatory disease. “Efficacy of Low Level Laser Versus Interferential in the Treatment of Chronic Inflammatory Disease” Bull. Fac. Ph. Th. Cairo Univ: Vol 11, No. (2). July 2009.; Yamato M, et al., concluding that externally directed LLLT (830 nm, 1×/day) on the shaved skin surface of rats suppresses the activity of anti-GBM crescentic glomerulonephritis. Lasers Med Sci. 2013 July; 28(4):1189-96. PMID: 23139073; M A W J, et al., concluding that LLLT (632 nm) has an anti-inflammatory effect on staphylococcus epidermis endophthalmitis in rabbits similar to dexamethasone. Lasers Med Sci. 2012 May; 27(3):585-91. PMID: 21948400; Saayman L, et al., concluding that LLLT as an adjunct for chiropractic joint manipulation (CMT) improves pain and range of motion management of cervical facet dysfunction. J Manipulative Physiol Ther. 2011 March-Ar; 34(3):153-63. PMID: 21492750; Mitchell U., concluding that nIFR LLLT significantly reduces symptoms associated with restless leg syndrome. Treatment consisted of 30 min. sessions, 3×/week for 4 weeks. The baseline score was 27 (severe). Patients were symptom free at week 2, and remained symptom free by week 4 symptom free. Symptoms slowly returned by week 3 following end of treatment. Journal of Medica. Case reports 2010, 4:286.; Hamblin M, et al., “Low Level Laser Therapy for Zymosan-Induced Arthritis in Rats: Importance of Illumination Time.” Lasers in Surgery and Medicine, 39:543-550 (2007).; Mitchell U. reporting the use of nIFR light to reduce the symptoms associated with restless leg syndrome. LLLT may revive the neglected vascular mechanism causing RLS. Journ of Med Case Reports. 2010; 4:286.; Yan W, et al., concluding that LLLT (650 nm, 808 nm) significantly relieves somatosensory and compound muscle action in rat sciatic nerves with implications for LLLT to induce analgesia for painful conditions. J Peripher Nery Syst. 2011 June; 16(2):130-5. PMID: 21692912.)

Aesthetic Applications

In some embodiments, the present disclosure contemplates a PB/LLLT handheld device with Green/Blue/Amber/Red/nIFR LEDs with optic enhancements for the application of light therapy in medical aesthetics. Medical aesthetics applications may include but are not limited to: acne, cellulite, photoaging/photorejuvenation, scar/skin care, facial/body contouring, PDT, and alopecia. PB/LLLT wavelengths for aesthetic applications may include Blue 492 nm (acne bacteria), Amber 590 nm (photo-aging, reduced scar formation), Red 660 nm (facial/body contouring, psoriasis, dermatitis, acne inflammation, photo-aging, Photodynamic Therapy), and nIFR 850 nm (facial/body contouring, photo-aging, Photodynamic Therapy). In some embodiments, a combination wavelengths for the treatment of specific conditions is provided. For example, acne vulgaris may require Blue 492 nm (bacteria) and Red 660 nm (inflammation). In some embodiments, the present disclosure provides light therapy for cellulite, androgenetic alopecia, and body contouring. Contrary to some embodiments of the present disclosure, conventional devices for alopecia comprise a comb requiring skin contact or helmets which cannot be handheld. In some embodiments, the present disclosure provides I Blue, Green and Amber as preferred wavelengths for Fitzpatrick skin types IV-V, or for heat sensitive conditions (e.g., eczema, rosacea). In some embodiments, persons with Fitzpatrick skin types IV-V respond with less sensitivity to the non Red/nIFR spectrum due their non-heating properties and are better able to meet their perceived needs or treatment application with these wavelengths. In other embodiments, Amber is a favorable wavelength for darker skin types (IV-V). In certain embodiments, the present disclosure provides Blue, Red, nIFR for bacterial infection, inflammation or PDT. In some embodiments, a treatment time of 1-5 mins/2″ diameter or 2.4 J/cm² to 4 J/cm² at a distance of 1″ from skin is preferred. In some embodiments, the present disclosure provides a device for the treatment of cellulite (e.g., Green LED), acne vulgaris (e.g., Blue/Red), facial/body contouring, photo-aging/rejuvenation, PDT (e.g., Green/Amber/Red/nIFR LEDs), and androgenetic alopecia (e.g., Red/nIFR LEDs) without need for surface contact. In other embodiments, the present disclosure provides LEDs for eczema, rosacea, Blue/Red/nIFR LEDs for bacteria/inflammation, and Red LED for psoriasis, virus related conditions. (See, for example: Jackson R F, et al., concluding that LLLT using Green (532 nm) diodes is safe and effective for improving the appearance of cellulite in thighs and buttocks. LLLT is effective as a stand-alone procedure without requiring massage or mechanical manipulation. Lasers Surg Med. 2013 March; 45(3):141-7. PMID: 23598376; Kim H, et al., concluding that LLLT as an effective treatment for androgenic aloplecia (AGA). The device used was a helmet with Red wavelengths (630,650,660 nm), and treatment time was 18 minutes daily. After 24 weeks, the LLLT group showed significant hair density and mean hair diameter improved statistically. Dermatol Surg. 2013 August; 39(8):1177-83, PMID: 23551662; Caruso-Davis M K, et al., concluding that Red (635-680 nm) LLLT achieves a safe and significant girth loss sustained over repeated treatments. In vivo studies suggest LLLT increases fat loss from adipocytes by release of triglycerides, without inducing lipolysis or cell lysis. Obes Surg. 2011 June; 21(6):722-9. PMID: 203930809.)

Dental Applications

PB/LLLT may be used in dentistry for tissue biostimulation (i.e., wound healing, bone graft formation), cleaning, postoperative pain management and TMJ/TMD applications. Conventional dental LLLT devices require an intraoral application and contact with skin, mucosa or other surface. In some embodiments, a handheld PB/LLLT device includes Red/nIFR LEDs and optic enhancements for dental applications without the need for contact of skin/bone or surface, or confined to intraoral use. In some embodiments, a device of the present disclosure may be held up to 0.5″ away externally from the cheek/jaw area and/or the light beamed into the oral cavity for treatment. In some embodiments, the present disclosure provides a device for the treatment of facial conditions wherein muscle stimulation is desired (e.g., temporomandibular joint pain). (See, for example: Chang P C, et al., concluding that Red (660 nm) light with 10 J/cm² is a suitable adjunct for periodontitis by reducing inflammation, facilitating collagen realignment and bundle bone formation. J Peridontal Res. 2013 April; 48(2):135-43. PMID: 22845797; Mazzetto M O, et al., concluding that LLLT is supportive therapy in the treatment of TMJ pain resulting in immediate decrease of painful symptoms and increased range of mandibular movement. Light therapy (830 nm, 40 mW, 5 J/cm²) was administered 2×/week for 4 weeks. Braz Dent J. 2010; 21(4):356-60. PMID: 20976388; Dostolova T, et al., concluding that LLLT is effective in improvement of the range of TMJ motion and promotes significant reduction of pain symptoms. LLLT (280 mW, 15.4 J/cm², 830 nm) was provided over five treatment sessions. Significant differences were observed in posterior and anterior face height. Unpleasant feeling on pain VAS was 27.5 to 4.16. Photo-medicine and Laser Surgery. 2012 May; 30(5):275-280.; Guarda M, et al., concluding that LLLT (860 nm, 70 mW, 4.2 J/cm² per point) in association with antibiotic therapy has a positive effect of tissue healing and remission of painful symptoms in the treatment of bisphosphonate-induced osteonecrosis of the jaw. Photomed Laser Surg. May 2012; 30(5):293-297.; Melchior Mde O, et al., concluding that LLLT is effective in decreasing subjective pain to palpation. Cranio. 2013 April; 31(2) 133-9. PMID: 23795403; Tanboga I, et al., concluding that

LLLT before cavity preparation decreases pain in pediatric dental patients. Eur Arch Pediatr Dent. 2011 April; 12(2):93-5. PMID: 21473840. Soares D M, et al., concluding that LLLT stimulates proliferation of a variety of cell types and has a positive stimulatory effect on the proliferation of hPDLSC (human periodontal ligament stem cells). Lasers Med Sci. 2013 Sep. 7. PMID 24013624.)

Men's Health Including Erectile Dysfunction and Fertility Applications

PB/LLLT increases release of nitric oxide and promotes vasorelaxation. Researchers at John Hopkins Medicine “found a complex positive feedback loop in the penile nerves that triggers waves of nitric oxide to keep the penis erect.” After the initial release of nitric oxide, the nerve impulses that begin in the brain or from physical stimulation are sustained due to a chemical process called phosphorylation. Blood vessels use the chemical as a signal to surrounding muscles to relax which increases blood flow—a necessity for staying erect. In some embodiments, a handheld PB/LLLT device is provided with Red/nIFR LEDs and optic enhancements for the treatment of erectile dysfunction and promotion of sperm motility requiring no skin or surface contact. (See, for example: Yacobi Y, concluding that the application of low level light induces vasorelaxation, which is the event that produces penile erection. Progress in Biomedical Optics and Imaging. 2001, vol. 2, nol, pp. 350-352. Application of light (808 nm, 150 mW, 20 mins, 2×/weeks, 6-8×) was externally applied to the penis/vascular bed of patients with erectile dysfunction (ED). Median Erectile Function domain score baseline score was 13 and increased to 20.5 after treatment (p=0.02). Many patients in the treatment group reported occurrence of morning erections.; Koultchavena E, et al., report that nIFR radiation (890 nm, 4 mW/cm², treatment time 5 mins., 1×/day) on penis erection glands reduces complaints of insufficient erection, premature ejaculation, and small size of the penis. After treatment, spontaneous erections were reported in all patients, adequate erections appeared sufficient for normal coitus (97.1%), and the time of coitus increased. Patients with micropenia through 2 months received a repeated rate of treatment with length of the penis in a quiet condition was increased from 12.5% to 33.3%). Revue/Journal Title Progress in biomedical optics and imaging ISSN 1605-7422 Source/Source 2001, vol. 2, no. 1, pp. 350-352.; Salman Yazdi R, et al., report that low level light irradiation on human sperm can improve their progressive motility. Fresh human semen specimens were treated with 808 nm light irradiation, 4/6 J/cm² with sperm motility assessed at 0, 30, 45 and 60 minutes post irradiation. Significant increases were observed at 45 and 60 minutes. Laser Med Sci. 2013 Feb. 14. PMID: 23407899.)

Neuromuscular Light Stimulation (NMLS) Applications

In some embodiments, the present disclosure provides a NMLS device comprising LEDs and optic enhancements as light source capable of neuromuscular stimulation similar to electrical stimulation as in a NMES, EMS or TENS device. In experiments conducted in the course of the development of the present disclosure, it was surprisingly discovered that light wavelengths are able to generate neuromuscular stimulation, with the depth and rate of stimulation and intensity of contraction dependent upon the wavelength. Many wavelengths of light have the ability to photo-stimulate the neuro/musculo-skeletal system and acupuncture/trigger points. Shorter wavelengths (e.g, Blue, Green, Amber) may produce more rapid contractions with shallow penetration versus longer wavelengths (e.g., Red, nIFR) with longer contractions and deeper penetration. In some embodiments, the present disclosure provides an NMLS device using transdermal application that targets, for example, a small nerve/muscle and motor neuron area or, for example, a larger nerve/muscle and motor neuron area by changing the distance from the skin, and provides specific rates and depths of muscle contraction by wavelength selection. In other embodiments, the present disclosure provides an NMLS device of use the field of optogenetics for application to slow and/or fast-twitch nerve fibers able to contract muscles and in preferred order. As referenced herein, “optogenetics” refers to neuromodulation techniques employed in neuroscience that provide a combination of techniques from optics and genetics to control the activity of individual neurons, and to precisely measure the effects of the manipulations in real-time.

In further embodiments, the present disclosure provides an NMLS device of use in the treatment of muscle weakness, atrophy, paralysis or conditions related to neurodegenerative/stroke disorders. In still further embodiments, the present disclosure provides an NMLS device for use in mitigating the effects of neurotoxins causing muscle paralysis. In some embodiments, the present disclosure provides a device of use in body contouring or reducing the overall circumference of treated body areas. In certain embodiments, a diversity of wavelengths alone and in combination are provided as muscle contractors. In particular embodiments, when a muscle is shortened or contracted during an application (e.g., isometric contraction) there is an increase in muscle toning or tightening, and a reduction in body circumference. In preferred embodiments, as the device contracts muscles, a user may stretch muscles and tendons away from the light source (PNF techniques) which increases range of motion and flexibility while increasing strength. In some embodiments, the present disclosure provides an NMLS device with Green, Blue, Amber, Red and nIFR LEDs with optic enhancements to promote body contouring, tightening and/or toning of a muscle by contracting the muscle or performing isometric contractions while shining the light source on the area, and range of motion, strength and flexibility when used alone or in conjunction with PNF techniques. In other embodiments, PB/LLLT devices of the present disclosure may enhance or reduce muscle fatigue or soreness, and may be used during exercise or as an alternative to exercise. In certain embodiments, by scanning or holding a PB/LLLT LED light source over the muscle(s) area, NMLS induces muscle contractions, increases ATP, releases NO, stimulates stem and progenitor cells, increases RBC, and reduces blood lactate, creatine kinase and C-reactive protein. In further embodiments, the present disclosure provides a handheld PB/LLLT device with Red/nIFR LEDs with optic enhancements applicable for enhancement and/or reduction of muscle fatigue or soreness that may be used as an adjunct or replacement therapy to increase physical training, rehabilitation or performance. (See, for example: Stanford School of Medicine and Engineering in a study published online Sep. 26, 2010 in Nature Medicine report light stimulation for optogenetic use). Investigators using Blue light stimulation provided by an “optical cuff” lined with LEDs placed around a sciatic nerve induced patterns of muscle contractions. Both slow and fast-twitch fibers were responsive with the proper firing sequence. Furthermore, light stimulated contractions were sustained longer and remained at plateau longer than by electrical stimulation. (Llewellyn M, et al. Nature Medicine. 2010 September; 16: 1161-1165.; Fontana C R, et al., concluding that LLLT (660 nm, 780 nm) applied unilaterally to 3 y/o boy suffering facial asymmetry due to Bell's Palsy resulted in improvement of facial movement and symmetry. J Altern Complement Med. 2013 April; 19(4):376-82. PMID: 23140111; Boonswang N A, et al., concluding that photobiomodulation demonstrates results in a subject experiencing dizziness, non-functional left hand (due to weakness), right hand severe spasticity, right lateral sixth nerve palsy and inability to ambulate due to a brainstem stroke. BMJ Care Rep. 2012 Sep. 11:2012. PMID: 22967677.; Nestor M, et al., concluding that LLLT (635 nm) is effective in reducing the overall circumference of specifically targeted regions, including the hips, waist, thighs and upper arms. Semin. Cutan Med Surg 2013 32:35-40, 2013; Leal E, et al., concluding that pre-exercise irradiation of the biceps with a 6 J/cm² (810 nm) light source increases elbow flexion and decreases post-exercise levels of blood lactate, creatine kinase, and C-reactive protein. “Effects of Low Level Laser Therapy in the Development of Exercise-Induced Skeletal Muscle Fatigue and Changes in Biochemical Markers Related to Postexercise Recovery.” Journal of Orthopaedic and Sports Physical Therapy. 2010 August; 40(8).; Ferraresi C, et al., concluding that LLLT is beneficial in muscle injuries with stimulation of stem/progenitor/muscle satellite cells, reduced inflammation and 3) lessened oxidative stress. Photonics Lasers Med., 2012 Nov. 1; 1(4):267-286. PMID: 23626925; De Almeida P, et al., concluding that Red and nIFR (660 nm, 830 nm) LLLT are effective in delaying the development skeletal muscle fatigue and enhancement of skeletal muscle performance. Lasers Med Sci. 2012 mar; 27(2):453-8. PMID: 21814736; Paolillo F R, et al., concluding that Red (634 nm) during treadmill training improved quadriceps powers and reduced peripheral muscle fatigue in postmenopausal women. Climacteric. 2013 Sep. 3. PMID: 23895414.)

Traumatic Brain Injury (TBI), Neuro-rehabilitation, Neurological and Neurodegenerative Disease Applications

Transcranial light therapy (TLT) is the application of LLLT to the scalp or head area. Most conventional devices use SLDs that are bulky in size, low in energy density, must be attached or 30 in contact to the scalp/forehead, have treatment times of 20-30 minutes, and offer a single wavelength (nIFR). Using conventional technology, TLT treatment area size is restricted to the face of the device and, if using a cold laser, requires grid or frequent point scanning which may induce thermal damage. In experiments conducted in the course of development of the present disclosure, it has been discovered that nIFR light may not provide the optimal penetration wavelength for certain areas of the head, and that Red LEDs penetrate equally or greater than nIFR. Accordingly, treatments with some embodiments of the present disclosure are less time-consuming (e.g., requiring just 5-10 minutes), are able to treat a larger area, avoid surface contact with distances up to 0.5″ for required dosage, and offer multiple wavelengths. In some embodiments, the present disclosure provides a TLT device for the application of Red/nIFR LEDs with optic enhancements for the treatment of TBI, neurological and neurodegenerative disorders and conditions, and for neuro-rehabilitation without surface contact, and with choices for selection of wavelength, energy density and time. In some embodiments, the present disclosure provides a TLT device for improvement of executive functioning and reduced mood lability. (See, for example: Rojas J C, et al., TLT is the application of low level light in the red-to-near-infrared wavelengths to modulate a neurobiological function or to induce a neurotherapeutic effect in a nondestructive and non-thermal manner. Biochem Pharmacol. 2013 Aug. 15; 86(4):447-57. PMID: 23806754; Hamblin, Michael, et al., Red to NIFR passes readily through the scalp or skull and can arrive at the cortical surface. One of the mechanisms of TLT is to prevent neurons from dying when they have been subjected to hypoxic, traumatic or toxic insults. A further mechanism is increased neurogenesis or the generation of neuronal precursors and birth of new cells. The two mechanisms of action of TLT promote application for stroke, traumatic brain injury and neurodegenerative disease. Photomed Laser Surg. 2011 July; 29(7): 443-446. PMCID: PMC3128319; Rojas J C, et al., showing that PB/LLLT: 1) increases the rate of oxygen consumption in the prefrontal cortex in vivo; 2) Extinction memory is enhanced; 3) fear renewal is reduced with reemergence of extinguished conditioned fear responses prevented; and 4) hermetic dose-response effects on the metabolic capacity of the pre-frontal cortex is induced. J Alzheimers Dis. 2012; 32(3): 741-52. PMID: 22850314; QuirkBJ, et al., concluding that nIFR LLLT delays disease progression in Parkinson's disease. Front Biosci (Elite Ed). 2012 Jan. 1; 4:818-23. PMID 22201916; “Role of LLLT in Neurorehabilitation,” NIHMSID:NIHMS281856. “Harnessing the cell's own ability to repair and prevent neurodegenerative disease.” NIHMSID:NIHMS55366. U.S. NIH clinical trial NCT01598532 with favorable results for SLDs placed on the scalp to improve working memory in people who have sustained mTBI. Gain was also found in psychological health with decreased mood lability along with improved executive functioning—Spaulding Rehabilitation Hospital/Harvard Medical Center, Dr. R. Zafonte, M D.

Psychological Applications

TLT may be used to treat a broader base of mood, anxiety, sleep and addiction disorders including Seasonal Affective Disorder (SAD). In some embodiments, the light source is applied to acupoints on the forehead/scalp, or light is scanned the over these same areas. In some embodiments, a TLT device with Green/Blue/Amber/Red/nIFR LEDs is provided with optic enhancements for the treatment of psychological disorders, or as an adjunct for therapeutic modalities (e.g., Prolonged Exposure Therapy, which is a form of behavior therapy and cognitive behavioral therapy used to treat, for example, post-traumatic stress disorder, characterized by re-experiencing the traumatic event through remembering it and engaging with, rather than avoiding, reminders of the trauma (triggers); the technique may also be referred to as flooding). In some embodiments, a device of the present disclosure provides equivalent benefit to electrical brain stimulation therapy for the treatment of psychological disorders. In certain embodiments, a device of the present disclosure provides RED wavelengths for anxiety disorders, and Red or Green wavelengths for depressive disorders. In further embodiments, Red/nIFR, Blue wavelength and Green wavelengths are used) for treatment of SAD and mood disorders. (See, for example: Tanaka Y, et al., report that infrared light exposure decreases indicators of depression and anxiety-like behaviors. The study found that the number of BrdU-positive cells in CAl of the hippocampus is significantly increased in both acutely and chronically exposed infrared irradiation groups. These results indicate that chronic infrared radiation may produce antidepressants- and anxiolytic-like effects. Brain Stimulation. Volume 4, Issue 2; 71-76, April 2011.)

Sleep Applications

In some embodiments, the present disclosure contemplates PB/LLLT promoting sleep wherein the ability to fall asleep may depend upon the duration and timing of treatment. (See, for example, Chang Y, et al. report that infrared exposure on acupoints increases serotonin levels for patients with insomnia. Am J Chin Med. 2009; 37(5):837-42.; Zhao J, et al., report the effectiveness of red light irradiation for improving sleep and serum melatonin levels. J Athl Train. 2012 November-dec; 47(6):673-8. McLean Hospital/Harvard Medical School has found blue light to improve sleep and cognition in mTBI patients. American Academy of Sleep Medicine. 2013, May 31. SLEEP 2013: Associated Sleep Societies 27th Annual Meeting. Abstract 0751. Presented Jun. 3, 2013.)

Regenerative Medicine and in vitro Applications Including Stem Cells, Bone Formation, Photodynamic Therapy and Combination Therapy Applications

In some embodiments of the present disclosure, PB/LLLT is provided for in vivo and in vitro applications. Currently, irradiation by Red/nIFR is used in transfusion medicine (blood), oncology medicine (bone marrow stem cells) and bone/tissue grafting. In some embodiments, PB/LLLT can be used alone, is used an activator in PDT or as an adjunct with drug combination therapy. In some embodiments, a handheld PB/LLLT device is provided with in vivo and in vitro applications configured to serve as a stand-alone or adjunct therapy with wavelength, energy density and application time choice. In some embodiments, the present disclosure provides a handheld PB/LLLT device for stem cell and regenerative medicine applications. In other embodiments, the as handheld PB/LLLT for irradiation of blood in transfusion medicine, bone marrow stem cells in oncology medicine, and other tissue/bone graft applications is contemplated. In certain embodiments, there is a handheld PB/LLLT device with Red (660 nm) for irradiation of adipose derived stem cells. In still further embodiments, there is a handheld PB/LLLT LED(Red/nIFR) device to activate and promote stem cell migration. (See, for example; Yazdani S O, et al., concluding that LLLT stimulates human Schwann cell (SCS) proliferation and NGF gene demonstration in vitro. SCS were stimulated by LLLT (810 nm, 50 mW, 4 J/cm²) with significant increase in proliferation on day 7. Photochem Photobiol B. 2012 Feb. 6; 107:9-13. PMID: 22178388; Gupta A, et al., concluding that a multimodal application of photobiology and nanotechnology can be used for the management of cancers, microbial infections or other tissue diseases, and promotes tissue repair and regeneration. Biotechnol Adv. 2013 September-October; 31(5):607-31. PMID: 22951919; Kim H, et al., concluding that LLLT is an effective stimulator of adipose-derived mesenchymal stem cells (ASCs), enhances the survival of ASCs and stimulates wound healing or growth factors in the wound bed. ASCs are an attractive cell source for skin tissue engineering and LLLT reduces ASC decline in recipient tissue. J Dermatol Sci. 2012 De; 68(3):149-56. PMID: 23084629; Abrahamse H, et al., concluding that hADSCs irradiated with 680 nm (Red) respond better than 830 nm (nIFR) wavelengths, and lower irradiation (5 J/cm²) responded better than higher (10, 15 J/cm²). Med Tech SA. 2010 December; 24(2): 15.; Gaasparyan L, et al., concluding that LLLT irradiation can activate stem cell migration. Laser Flor. 2004.; Hou J F, et al., concluding that LLLT stimulates proliferation, growth factors secretion and facilitates myogenic differentiation of bone marrow mesenchymal stem cells (BMSCs). LLLT may provide preconditioning of BMSCs in vitro prior to transplantation. Lasers Surg Med. 2008 December; 40(10):726-33. PMID: 19065562.; Soares L G, et al., conclude LLLT is effective for enhancing new bone formation with consequent increase of bone-implant interface in both autologous grafts and xenografts. Braz Den J. 2013 May-June; 24(3):218-223.)

LLLT Intravenous Irradiation (LLLT II) Applications

In some embodiments, there is provided an LLLT Intravenous Irradiation comprising low intensity light via transdermal application directly over an artery or vein, or intra-nasally by shining light on blood vessels inside the nose, to stimulate the immune system, improve blood circulation, reduce inflammation, and to activate cell metabolism and/or the production of new cells. In this fashion, the vascular blood network is receptive to external stimulation that is then circulated in the body's circulatory system. (See, for example: Babaev A V, et al., reporting a course of LLLT II treatment to alleviate clinical symptoms and significant positive changes in biochemical parameters (AST, ALT, bilirubin, alkaline phosphatase, lactate dehydrogenase and total cholesterol). Bull Exp Biol Med. 2012 September; 153(5):754-7.; Yamaikina I, et al., report rheological changes in blood and plasma following transdermal irradiation of the tail vein of rats (806 nm, 2 j/cm²). Observed rheological changes arose from the removal of irradiation-damaged erythrocytes in the blood channel of young and highly deformable red cells. J of Engin Phys and Thermophys. 2012 30 May; 85(3):655.

“Body Washing” Applications

In some embodiments, the present disclosure provides a handheld PB/LLLT (Red/nIFR) device for use by HIV(+) and other immune-deficient clients to increase CD4 levels with and/or without retroviral drugs, and to decrease viral load without retroviral drugs. (See, for example: Halon A, et al., concluding that application of LLLT (820 nm, 200 mw, 6 J/cm²) for the treatment of tooth extraction wounds in HIV(+) patients enhances the formation of new blood vessels, which in turn promotes wound healing. Lasers Med Sci. 2013 Aug. 6. PMID: 23917415.)

Diabetes Applications

In some embodiments, a handheld PB/LLLT (Red/nIFR) device is provided for irradiation treatment to ameliorate diabetes. (See, for example: Peplow P V, et al., reporting that nIFR (810 nm, 50 mW) irradiation of the left inguinal region of diabetic mice 1) reduces body weight and water intake by Day 7, and 2) blood plasma fructosamine levels are significantly lower. The study concludes that irradiation of the left inguinal region with 810 nm LLLT may ameliorate diabetes. Lasers Surg Med. 2013 April; 45(4):240-5. PMID: 23568826.)

Cancer and Radiotherapy Applications

In some embodiments, the compositions, methods, systems and kits of the present disclosure provide a handheld LLLT device that reduces the deleterious impact of radiotherapy, and increases the Quality of Life (QOL) of patients with cancer. (See, for example: Oton-Leite A F, et al., concluding that LLLT reduces the impact of radiotherapy on the QOL of patients with head and neck cancer. The LLLT group showed positive QOL scores regarding pain, chewing and saliva domains. Head Neck. 2012; 34(3):398-404. PMID: 21472883

Acupuncture, and Myofascial (MYF) Trigger Point Applications

In some embodiments, a handheld PB/LLLT device is provided for acupuncture and MYF trigger point application. Diverse wavelengths have the ability to stimulate points depending on wavelength and energy density. Deeper points may be triggered with Red/nIFR wavelengths. In some embodiments, point stimulation is 1-2 minutes per point without surface contact. In other embodiments, the present disclosure provides a handheld PB/LLLT device for a heating skin area comprising inserted needles. Warming the regions and point may stimulate circulation, and induce a smoother flow of blood and qi. As used herein “Qi” (also “chi” or “ch′I”) is an active principle forming part of a living thing. Qi may be translated as “natural energy”, “life force”, or “energy flow”. Qi is a central underlying principle in traditional Chinese medicine. Literal translations of “qi” include “breath”, “air”, or “gas.” In certain embodiments, the present disclosure provides a handheld PB/LLLT device configured for application to Ah Shi point(s). (See, for example: Zhou G Y reporting that LLLT irradiation on acupoints increases the audiometry and auditory function of moderate and severe sudden deafness. Zhongguo Zhen Jiu. 2012 May; 32(5):413-6.; Moustafa Y, et al., concluding that LED phototherapy for children with allergic rhinitis shows significant improvement in severity scores through and by the end of the follow-up period (1 year). Int J Pediatr Otorhinolaryngol. 2013 May; 77(5):658-65. PMID: 23394792.)

Veterinary Applications

In some embodiments, a handheld PB/LLLT device is contemplated for veterinary applications. (See, for example: Draper W E, et al., concluding that LLLT (830 nm, 200 mW) in combination with surgery decreases the time to ambulate in dogs with T3-L3 myelopathy secondary to intervertebral disk herniation. J Small Anim Pract. 2012 August; 53(8):465-9. PMID: 22783835.; Dadone L I, et al., concluding that LLLT with cervical range of motion exercises reduces cervical muscle hypertonicity significantly with a male 2 y/o giraffe with severe acute-onset torticollis. J Zoo Wildl Med. 2013 March; 44(1):181-5. PMID: 235005724.; Bellows J. reports tht LLLT is useful for oral surgery in veterinary dentistry. Vet Clin North AM Small Anim Pract. 2013 May; 43(3):651-68. PMID: 23643025.)

PB/LLLT Compositions

In some embodiments, there is a device configured to simultaneously 30 provide two or more wavelengths of PB/LLLT to a shared area, for example, Blue and Red wavelengths in the treatment of acne. In certain embodiments, the two or more wavelengths provide different penetration depths to the same treatment area. In other embodiments, wavelengths are combined to provide increased energy density at a short distance from a surface (e.g., 0.25″), and/or to maintain energy density with increased distances from a surface. In still further embodiments, interference of combined wavelengths (i.e., Inferential Therapy) provides a reinforced wavelength through interference. (See, for example, Montes-Molina R, et al., concluding that interference is a significant feature for treatment of musculoskeletal pathologies. Physiotherapy. 2012 June; 98(2):143-50. PMID: 22507365.; Montes-Molina R, et al., concluding that interferential light therapy or the effect of two light probes (600 nm, 900 nm) is a safe and effective regarding shoulder pain reduction during abduction and external rotation movements. Clin Rehabil. 2012 December; 23(12):1114-22. PMID:22643725.) In certain embodiments, combined wavelengths are used to raise exposure to tissue temperatures of 40-45° C. to 30 seconds or less. In some embodiments, output beams from one or more LEDs are aligned to overlap in order to improve efficacy in a treatment area. In other embodiments of the present a LED heat-sinked an fan, and optic assembly (e.g., reflectors and diffusing lens) are rotated 15 degrees from vertical so that their respective emitted light overlaps between 0.25″ and greater from surface contact. In particular embodiments, the present invention comprises a device with input power of >1 W and <10 W with integrated optic assembly units. In preferred embodiments, output power is 500 mW or less per discrete LED to improve efficacy in a treatment area. In some embodiments, the present invention comprises a battery. In further embodiments, the present disclosure provides wavelength selection between 400 nm and 1000 nm.

Integrated optics may enhance light intensity due to reduced dissipation, and allow a user to control the area and size of treatment. In the case of interferential therapy with multiple emission wavelengths detailed above, the integrated optics are understood to improve penetration of the shorter wavelength. In addition, phototherapy units with integrated optics are less of a health contamination issue because the device does not touch the skin.

In some embodiments, a belt and/or holding fixture may attach the device to an object, for example, to a pillow or nightstand/table. In specific embodiments, the present disclosure is pole mounted to easily reach posterior surfaces. In other embodiments, the present disclosure comprises replaceable/interchangeable LED modules configured for the user to select the preferred light frequencies for a particular treatment. For example, doped silica LEDs have narrow emission bands (single color), and alternative frequencies may be better suited for various treatment methods in other embodiments. In further embodiments, the present invention comprises onboard or outboard rechargeable batteries for non-tethered operations. In other embodiments, optics lens, reflectors and other optical enhancements are provided for a shallower or broader emission angle, thereby allowing treatment of larger areas at lower power, and limiting the need to “scan” the unit over a broad treatment area. In particular embodiments, the present invention provides an adjustable reflector to adjust beam width during treatment. In some embodiments, the application the beam may be tightly focused for spot treatment, or broadened for area treatment in other embodiments. In some embodiments, the device is shielded from radio-frequency and other ambient radiation. In other embodiments, the device is shielded from emitting radio-frequency or undesired other radiation.

In some embodiments, the compositions, methods, systems and kits of the present invention provide a photo-voltaic sensor on an emitter face to measure light reflecting from active LEDS on a user's skin or body surface. In other embodiments, the intensity of this signal is used to calculate exposure range, and to provide an audible tone and alarm if exposure distance targets are beneath or beyond a target range. In other embodiments of the present invention, physical contact probes are affixed to the emitter face. In this fashion, so when a user treats non-visible body surfaces, direct contact between the surface and the probes to indicate distance, thereby informing the user of the orientation of treatment areas via tactile feedback over surfaces that cannot be seen. In further embodiments, the present disclosure provides a disposable lens attached to an emitter face allowing the operator or user to press against a treatment area at an exact, pre-determined distance without risk of contamination. In some embodiments, the lens is a disposable lens.

In some embodiments, there is provided instructional material to users that describe techniques for using the hand held unit. For example, visual instructions provide tutorials on using the product without having to read extensive text. In some embodiments, instructions are provided upon computer readable media. In other embodiments, instructions are provided upon the World Wide Web. In certain embodiments, the present invention provides a menu for the user to select a particular malady with instructions for the method and duration of treatment necessary to treat that malady. In other embodiments, the present disclosure provides a diary function to record treatment duration. In some embodiments, uploading this data provides the prescribing healthcare professional and user the ability to track usage and physiological change over an extended treatment period.

In some embodiments, the present invention comprises a computer-based application that provides direct control of user options. For example, a user or operator selects a given malady and a corresponding application pre-programs an embodiment of the present disclosure with preferred treatment duration LED wavelengths to best treat that malady. In other embodiments, the present invention is configured to allow multiple users to login and diary their individual treatments separately. In certain embodiments, an application of the present invention provides an audio interface for the visually impaired. In further embodiments, the present disclosure comprises an avenue to update/upgrade firmware in a handheld device of the present description. In still further embodiments, the present disclosure comprises an application configured to allow users to communicate with PB/LLLT users to share experience and discuss treatment experiences.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present disclosure.

### EXPERIMENTAL EXAMPLES

Photobiology devices in accordance with the present invention were used to evaluate

effectiveness of treatment for a variety of disorders. Subjects were volunteers who were uncompensated for their participation. Application times were from 1 to 5 minutes for total body use and did not exceed two treatment sessions per day. Volunteers wore protective eye goggles similar to those used in UV tanning beds and were advised to remain well hydrated after each treatment.

In one application of photobiology treatment employing methods and devices of the present invention, a female subject in her mid-twenties presented with complaints of osteorthritis and rheumatoid arthritis, with pain in right hip requiring the use of crutches to walk. Phototherapy treatments once and twice daily using a device of the present invention lasted 1-5 minutes. Thereafter she reported the ability to walk without crutches, and with greatly reduced pain after one treatment. After one week, she reported an increase in muscle mass, strength and no pain. In another application of photobiology treatment employing methods and devices of the present invention, a male subject in his mid-sixties presented with complaints of chronic knee pain and inability to exercise. Treatment was applied via high-powered LED light from an apparatus in accordance with the present invention. Treatment sessions of 1 to 5 minutes each were applied to both knees. He reported greater flexibility and reduced pain thereafter. Continued use of the device of the present invention, and treatment for two weeks, produced improved strength and ability to resume exercise.

Similar treatments with the compositions, methods and devices of the present invention, have benefited patients suffering from knee injuries, osteoarthritis, chronic pain, anxiety, muscle tone, sciatica, bursitis, mouth sores, lower back pain, hip and leg pain, athlete's foot, carpal tunnel

syndrome, bladder dysfunction, arthritic pain in hands and feet/ankles, ankle or joint sprain, knee pain, sports injuries, hemorrhoid, warts, cold sores, scoliosis, lower back pain, migraine headache, eczema, shingles, neuropathic pain, hematoma, skin ulcers, psoriasis, and pressure sores.

Transdermal Experimental Examples

Male (61 y/o), shingles rash with pain. Tx: Red 660 nm., 1″ from skin, 1 min per 2″ diameter, 4 J/cm², 1×/day. Results: rash and pain resolved 2 days of tx. NOTE: tx with 850 nm was not effective for rash or pain.

Male (91 y/o), shingles rash with pain. Tx: Red 660 nm, 1″ from skin, 1 min. per 2″ diameter, 4 J/cm², 1×/day. Results: rash and pain resolved 3 days.

Female (25 y/o), herpes simplex type 1 blister, crust stage. Tx: Red 660 nm, 1″ from skin,

1 min., 4 J/cm², 1 tx. Results: blister resolved 2 days after tx.

Male (20 y/o), HIV positive, herpes simplex type 1 blisters, prodromal stage. Tx: Red 660 nm, 1″ from skin, 1 min per blister, 4 J/cm², 1 tx. Results: blisters resolved 3 days after tx. NOTE: 850 nm was not effective.

Female (55 y/o), psoriasis and pain. Tx: Red 660 nm, 1″ from skin, 1 min. per 2″ diameter, 4 J/cm²., 2×/daily for 3 days. Results—skin tone inflamed to normal, reduced pain, “itching,” and scaling. NOTE: 850 nm was not effective on skin condition with minimal reduction of pain.

15 Male (23 y/o), IED blast victim, both lower limb amputation, numerous scar tissue and pain from 72 surgeries including skin grafts, wound lesion from prosthetics. Tx: 660 nm, 1″ from skin, 1 min. per 2″ diameter, 4 J/cm²., 1× daily 14 days. Results” wound lesion healing within 3 days, scar tissue “flattening,” normal pigmentation including freckles on scar tissue with 14 days, pain level 0.

Pain and Inflammation Examples

Male (23 y/o), phantom limb and neuropathic pain in amputated stumps. Tx: Red/nIFR, 5 mins. 2/day, 7 J/cm², 1″ from skin, pain VAS 7. Results: by day 3, pain VAS 0 for phantom limb and 25 neuropathic pain. Stopped taking opiates by week 3.

Male (19 y/o), dislocated shoulder (wrestling) with range of motion 30%. MD examination was no wrestling activities for 8 weeks with possible surgery. Tx: Red/nIFR 5 mins. 2×/day, 7 J/cm² for 1 week, 1×/day 2 weeks, pain VAS 7. Results: after 3 days, pain VAS 2. At 3 weeks 30 cleared to return wrestling with full range of motion and no surgery.

Male (31 y/o), male professional bodybuilder, chronic back lateral muscle strain, unable to raise arms beyond shoulder level, disrupted sleep due to pain and unable to use computer work more than 15 minutes without pain. Tx: Red/nIFR 5-10 mins. 2×/day for 10 days, 1×/day for 1 week. Results: day 1, able to sleep through night w/o pain, able to use computer 1 hour w/o pain. Day 10: able to return to lifting weights overhead. Day 21: using LLLT as PRN for pain relief. Stopped taking analgesics by week 2.

Male (33 y/o), MMA fighter with elbow pain post-surgery. Tx: Red/nIFR, 5-10 mins 2×/day for 3 days. Pain VAS 7. Results: day 3, pain VAS 1.

Female (50 y/o), inflammation and pain from post-surgical metal pin implant for torn finger tendon. Tx: Red, 2-3 mins. 2×/day. Results: day 2 reduced inflammation/pain and able to use finger (with splint) at work.

Male (33 y/o), restless leg syndrome, disrupted sleep, chronic daily pain, severe leg movement during night. Tx: Red/nIFR, 5 mins, 10 J·cm², 2×/day, 1 week; 1×/day 2 weeks. Results: week 1: reduction in sleep disturbance, pain and leg movements. Week 3: no pain, no movements during sleep and able to resume Yoga for ongoing management. PRN when symptoms reoccur.

Aesthetic Application Examples

Female (55 y/o), psoriasis and pain. Tx: Red 660 nm, 1″ from skin, 1 min. per 2″ diameter, 4 J/cm²., 2×/daily for 3 days. Results—skin tone inflamed to normal, reduced pain, “itching,” and scaling. NOTE: 850 nm was not effective on skin condition with minimal reduction of pain.

Female (60 y/o), eczema on scalp, face, in ear canal and facial rosacea. Tx: Amber 590 nm, 2.4 J/cm², 3 mins. per 2″ tx area, 1″ from skin, 1×/day, 2 days. Results: eczema resolved, reduced “itching,” and rosacea reduced 80%. Both conditions return 1 week post treatment application. Note: Red application increased sx's and rash for both eczema and rosacea.

Male (12 y/o, African-American skin), eczema under chin, inner elbow and knee creases. Tx: Amber 590 nm, 2.4 J/cm² per 2″ tx area, 1″ from skin, 1×. Results: reduced itching and rash.

Female (55 y/o, Asian skin), eczema inner elbow crease. Tx: Amber 590 nm, 2.4 J/cm² per 2″ tx. area, 1″ from skin, 1×. Results: reduced itching and rash. Note: Red application increased sx's and rash.

Female (44 y/o), facial rosacea. Tx: Amber 590 nm, 2.4 J/cm² per 2″ area, 1″ from skin, 1×. Results: rosacea reduced 90%. Facial rosacea returned 1 week post treatment. Note: Red application increased rosacea.

Male (57 y/o), facial rosacea. Tx: Amber 590 nm, 2.4 J/cm² per 2″ area, 1″ from skin 1×. Result: rosacea reduced 80%. Facial rosacea returned 1 week post treatment.

Dental Applications

Female (55 y/o): 1) bone graft for dental implant. Tx: Red (660 nm) and nIFR (850 nm), scanning cheek area ¼″ above bone graft side, 4 mins 2×/day for 1 week, 1×/day for 1 week. Result: bone graft healing 50% faster (1 month versus 2 month). 2) Wisdom tooth extraction, drug sensitivity/allergy. Tx: Red (660 nm) and nIFR (850 nm), scanning cheek area ¼″ above extraction site, 3 mins. 1×/day for 1 week. Results: no bruising, swelling or pain. Wound healing and suture removal 50% faster.

Female (40 y/o), nerve pain following root canal 1 week prior. Tx: Red (660 nm), scanning cheek area ¼″ above site, 3 mins. 1×. Result: pain 0.1 hour post treatment.

Men's Health Applications

Male (44 y/o), hx for ED. Tx: 5 mins, scanning, 660 nm, 4 J/cm², 1×tx. Results: reported return_of early morning erection and perceived increase in girth (diameter).

Male (61 y/o). Tx: 5 mins. scanning, 660 nm, 4 J/cm² 1×tx. Results: perceived increased girth (diameter).

NMLS Applications

Female (65 y/o), facial paralysis (left side) post stroke event. Tx: Red 660 nm, 3 mins, 4 J/cm² scanning lower face half, 1×. Results: facial symmetry with facial muscles/appearance matching non stroke side. NOTE: application of light to hand improved post stroke related pain.

Female (55 y/o), botox to forehead 2 weeks prior, unable to move muscles. Tx: Red 660 nm, 3 mins. scanning forehead and scalp area, 1×/day, 1 week. Result: day 7, able to regain 90% of muscle movement.

15 Male (19 y/o), (1) scanning of shoulder and upper arm area prior to shot-put meet (5 mins, Red/nIFR, 4-7 J/cm², 1×). Results: longest distance throw with increased fast-twitch muscle response. (2) post workout lactic acid pain, scan of thigh muscles (5 mins, Red, 4 J/cm2, 1×). Results: 15 mins after application, pain reduced.

Male (31 y/o), professional bodybuilder, scanning of muscles prior to workout (5 mins Red/nIFR, 5 mins per muscle area, 4-7 J/cm², 1×). Results: able to extend workout with reduced fatigue and soreness afterwards. Able to “sculpt” muscles and extend workouts to date of contest rather than stopping 6 weeks before as standard practice.

Male (65 y/o), bicyclist with chronic knee pain. Tx: bending/straightening knee muscles during light application. Red/nIFR, 5 mins per knee. Results: increased tone and perceived equivalence to 1 mile bike ride uphill.

TLT, Psychological, Sleep Applications

Male (23 y/o): Wounded Warrior sustaining TBI from IED blast with two amputated lower limbs. Tx: scan forehead/scalp areas (acupoints as in research), 3-4 mins. total treatment time per day, 660 nm, ½″ from skin. Result: after 2 days significant decrease in mood lability (anger/irritability, anxiety/depression) and improved executive functioning. Sleep insomnia was also reduced with increased ability for extended night-time sleep.

Male (30 y/o): social anxiety dx., score of 7 (1-10) anxiety sxs when entering restaurants, large social situations. Tx: scan forehead/scalp, Red 660 nm, ½″ from skin, 3 mins. total tx time. Result: entering/eating restaurant 30 mins. after tx, score of 1.

Male (31 y/o): anxiety with large dogs, score of 8 (1-10) anxiety sxs. Tx: scan forehead/scalp, Red 660 nm, ½″ from skin, 3 mins. total tx time. Result: 45 mins. after tx, score of 2 when introduced to and walking dog.

Male (44 y/o): major depression with poor response to anti-depressants, feelings of lethargy and

poor sleep. Tx: 660 nm, 3 minutes scan over forehead, 4 J/cm², 1× daily for 1 week. Results: end of day 1, reported elevation of mood and reduction in depression, increased energy and ability to engage/complete ADLs, and increased night-time sleep. End of week 1, continued mood, sleep and energy improvement. Note: when tx was stopped, did return to baseline 1 week post termination of light therapy.

For example, when light is applied 1-2 hours before normal bedtime, the ability to sleep may be disrupted. Application by 6 subjects 1-2 hours before normal bed time found the ability to sleep disrupted by 4-5 hours due to increased mental stimulation and alertness. Light applied 3-4 hours prior to normal bedtime did not disrupt sleep and will aid in sleep.

Body Washing Applications

Male (20 y/o), HIV (+). Tx: no retroviral drugs. 1×, 660 nm/850 nm, 1″ from skin, 7 mins. lower 30 abdominal area, alternating months 1-3 days prior to lab workup. Results: during months (2) treated with LLLT, lab results showed 22-25% increase in CD4 levels and 20% reduction in viral load. Tx: with retroviral drugs, same dose alternating months. Results: month (1) with LLLT, 25% increase in CD4 levels, no change in viral load.

Acupuncture Applications

Female (44 y/o), injured Achilles tendon, painful Ah Shi point(s). Tx: 660 nm, 1 min, 1×, 1″ above skin. Result: release of Ah Shi point with pain level of 8 to 1. As used herein, an “Ah shi tender point(s)” may be found in painful diseases. Ah shi point(s) may be felt as a pea-sized nodule(s) under the skin.

Veterinary Applications

Bulldog: arthritis joints, unable to stand without assistance. Vet consult, surgery. Tx: 5 mins Red (660 nm), 1×/daily to both sides of joints and along muscles for 3 days. Results: dog able to get up, walk and gained 95% of prior ADL. Vet consult, no surgery required.

German Shepherd: 1) panosteitis, lameness in both front legs, unable to go up/down stairs. Rimadyl for pain management. Tx: 3 mins. Red/nIFR 2×/daily, 3 days, scanning muscles/bone. Results: dog able to go/up down stairs, lameness gone, Rimadyl terminated. Tx continued on a PRN basis. 2) total hip replacement. Tx: 3 mins Red/nIFR, 2× daily with first tx immediately after surgery. Result: no bruising, swelling, wound healing 50% faster, reduced muscle atrophy with faster recovery time. 3) slippery paws, unable to walk on slippery surfaces, splayed paws, unable to grip floors, weak front leg muscles with hyperextension. Tx: 3 mins. Red/nIFR along front leg muscles and on bottom of paws to induce contractions 2×/day. Result: day 7, able to walk on slippery surfaces, able to grip paws and no hyperextension of legs. 4) dermatitis. Tx: 1 min Red 660 nm 2×/daily. Result: day 2 dermatitis resolved.

Dachshund: herniated disc surgery. Tx: 2 mins Red 660 nm, 2×/daily along surgical area. Result: wound healing 50% faster, no pain noticed with dog able to ambulate sooner.

What is claimed is:

1. A light emitting diode photobiology device for treatment of biological tissues, the device comprising:
a plurality of light emitting diodes, a first one of the plurality of light emitting diodes having a first predetermined wavelength with a first emission axis and a second one of the plurality of light emitting diodes having a second predetermined wavelength with a second emission axis;
a plurality of optics including a first optic corresponding to the first one of the plurality of light emitting diodes that defines a first dispersion pattern of enhanced light intensity centered on the first emission axis, and a second optic corresponding to the second one of the plurality of light emitting diodes that defines a second dispersion pattern of enhanced light intensity centered on the second emission axis; and
an optical face defined by a flat planar surface;
wherein the first one of the plurality of light emitting diodes is positioned in a first tilted angular relationship relative to the flat planar surface of the optical face and the second one of the plurality of light emitting diodes is positioned in a second tilted angular relationship relative to the flat planar surface of the optical face, the first emission axis and the second emission axis intersecting at a predefined distance away from the optical face and defining a substantially overlapping emission region of the first dispersion pattern and the second dispersion pattern.

2. The device of claim 1, further comprising:
a housing, with which the optical face is integrated, the housing enclosing the plurality of light emitting diodes and the plurality of optics.

3. The device of claim 2, wherein the housing is defined by a handle portion with a top surface and an emission portion including the optical face.

4. The device of claim 3, further comprising:
a user control area including indicators and switches by which a user selects and confirm desired treatment parameters.

5. The device of claim 1, wherein the optical face includes a diffuser for improving uniform dispersion of light.

6. The device of claim 1, wherein either one or both of the first tilted angular relationship and the second tilted angular relationship to the optical face has a quantified range between 1 degrees and 45 degrees.

7. The device of claim 1, further comprising:
a first emitter housing receiving the first one of the plurality of light emitting diodes and the corresponding first one of the plurality of optics therefor, the first emitter housing maintaining the first tilted angular relationship relative to the flat planar surface and fixed to the housing; and
a second emitter housing receiving the second one of the plurality of light emitting diodes and the corresponding second one of the plurality of optics therefor, the second emitter housing maintaining the second tilted angular relationship relative to the flat planar surface and fixed to the housing.

8. The device of claim 1, wherein angles of dispersion of the light emitting diodes is between 45 degrees and 90 degrees.

9. The device of claim 1, wherein an input power rating of either or both of the first and second ones of the plurality of light emitting diodes is greater than 1 watt and less than 10 watts.

10. The device of claim 1, wherein one of the plurality of light emitting diodes is selected from a group consisting of red diodes, near infrared diodes, and amber diodes.

11. The device of claim 1, wherein one of the plurality of light emitting diodes is an amber diode emitting light at a wavelength of 590 nm.

12. The device of claim 1, wherein one of the plurality of light emitting diodes is a red diode emitting light at a wavelength of 660 nm.

13. The device of claim 1, wherein one of the plurality of light emitting diodes is a near infrared diode emitting light at a wavelength of 850 nm.

14. A portable, high-powered light emitting diode photobiology device for use in phototherapy applications and treatment of biological tissues, the device comprising:
a plurality of light emitting diodes, each of said light emitting diodes having an input power rating greater than 1 and less than 10 watts and a preselected angle of tilt;
a plurality of optics, each optic comprising a reflector, associated with one of said light emitting diodes and providing dispersion angles of 45-90 degrees, for enhancing light intensity;
a user control area providing indicators and switches by which a user may select and confirm desired treatment parameters;
a housing substantially enclosing and retaining said light emitting diodes, optics and user control area; and
an optical face substantially integrated with said housing to provide a smooth surface toward the area of treatment, said optical face comprising a diffuser for uniform dispersion of light.

15. The device of claim 14, wherein one of the plurality of light emitting diodes is selected from a group consisting of red diodes, near infrared diodes, and amber diodes.

16. The device of claim 14, wherein one of the plurality of light emitting diodes is an amber diode emitting light at a wavelength of 590 nm.

17. The device of claim 14, wherein one of the plurality of light emitting diodes is a red diode emitting light at a wavelength of 660 nm.

18. The device of claim 14, wherein one of the plurality of light emitting diodes is a near infrared diode emitting light at a wavelength of 850 nm.

19. The device of claim 14, where each of the plurality of light emitting diodes are simultaneously activatable.

20. The device of claim 19, wherein each of the plurality of light emitting diodes define a respective dispersion pattern, a totality of each of the dispersion patterns defining a substantially overlapping emission region.
